SEQUENCE LISTING (40 sequences)

SEQ ID NO: 1 - DNA, 10 bp, SARS-CoV-2
  misc_feature (241)..(250): Mutation C241A, 5′UTR referred to as COIN-CoV2- 241
agtccgggtg

SEQ ID NO: 2 - DNA, 10 bp, SARS-CoV-2
  misc_feature (3031)..(3040): Mutation C3037T, ORF1ab referred to as COIN- CoV2-3037
ttctttttac

SEQ ID NO: 3 - DNA, 10 bp, SARS-CoV-2
  misc_feature (14401)..(14410): Mutation C14408A, ORF1ab referred to as COIN- CoV2-14408
ttcccacata

SEQ ID NO: 4 - DNA, 10 bp, SARS-CoV-2
  misc_feature (28141)..(28150): Mutation T28144C, ORF8 referred to as COIN- CoV2-28144
gttcaccttt

SEQ ID NO: 5 - DNA, 10 bp, SARS-CoV-2
  misc_feature (23401)..(23410): Mutation A23403C, Protein S referred to as COIN-CoV2-23403
ggctgttaac

SEQ ID NO: 6 - DNA, 10 bp, SARS-CoV-2
  misc_feature (28881)..(28890): Mutation G28881A, Protein N referred to as COIN-CoV2-28881
agggaacttc

SEQ ID NO: 7 - DNA, 10 bp, SARS-CoV-2
  misc_feature (28881)..(28890): Mutation G28882T, Protein N referred to as COIN-CoV2-28882
gtggaacttc

SEQ ID NO: 8 - DNA, 10 bp, SARS-CoV-2
  misc_feature (28881)..(28890): Mutation G28883A, Protein N referred to as COIN-CoV2-28883
ggagaacttc

SEQ ID NO: 9 - DNA, 10 bp, SARS-CoV-2
  misc_feature (17741)..(17750): Mutation C17747T, Protein NSP13 referred to as COIN-CoV2-17747
gtaaccttgc

SEQ ID NO: 10 - DNA, 10 bp, Influenza A virus H1N1
  misc_feature (231)..(240): Mutation C238A, ORF referred to as COIN-IFA-238
gatcctgaga

SEQ ID NO: 11 - protein, 10 aa, Influenza B virus Victoria
  MISC_FEATURE (129)..(138): Mutation at D129G of the hemagglutinin (HA) protein of influenza B viruses (“B/Victoria/2/87-like”)
GENIRLSTSN

SEQ ID NO: 12 - protein, 10 aa, Influenza B virus Victoria
  MISC_FEATURE (490)..(499): Mutation at R498K of the hemagglutinin (HA) protein of influenza B viruses (“B/Victoria/2/87-like”)
SAVEIGNGKF

SEQ ID NO: 13 - DNA, 22 bp, Artificial Sequence
  SYBR Green SARS-COV2-FOR Primer 1
  misc_feature (1)..(22): SYBR Green SARS-COV2-FOR Primer 1
gcctcttctcgttcctcatcac

SEQ ID NO: 14 - DNA, 19 bp, Artificial Sequence
  SYBR Green SARS-COV2-REV Primer 1
  misc_feature (1)..(19): SYBR Green SARS-COV2-REV Primer 1
agcagcatcaccgccattg

SEQ ID NO: 15 - DNA, 23 bp, Artificial Sequence
  SYBR Green SARS-COV2-FOR Primer 2
  misc_feature (1)..(23): SYBR Green SARS-COV2-FOR Primer 2
agcctcttctcgttcctcatcac

SEQ ID NO: 16 - DNA, 19 bp, Artificial Sequence
  SYBR Green SARS-COV2-REV Primer 2
  misc_feature (1)..(19): SYBR Green SARS-COV2-REV Primer 2
ccgccattgccagccattc

SEQ ID NO: 17 - DNA, 22 bp, Artificial Sequence
  Dual Labeled probe 1 SARS-COV2-FOR Primer 1
  misc_feature (1)..(22): Dual Labeled probe 1 SARS-COV2-FOR Primer 1
ccgcaaggttcttcttcgtaag

SEQ ID NO: 18 - DNA, 25 bp, Artificial Sequence
  Dual Labeled probe 1 SARS-COV2-REV Primer 1
  misc_feature (1)..(25): Dual Labeled probe 1 SARS-COV2-REV Primer 1
tgctatgtttagtgttccagttttc

SEQ ID NO: 19 - DNA, 25 bp, Artificial Sequence
  Dual Labeled probe 1
  misc_feature (1)..(25): Dual Labeled probe 1
aaggatcagtgccaagctcgtcgcc

SEQ ID NO: 20 - DNA, 20 bp, Artificial Sequence
  Dual Labeled probe 2 SARS-COV2-FOR Primer 2
  misc_feature (1)..(20): Dual Labeled probe 2 SARS-COV2-FOR Primer 2
ggcttaccgcaaggttcttc

SEQ ID NO: 21 - DNA, 25 bp, Artificial Sequence
  Dual Labeled probe 2 SARS-COV2-REV Primer 2
  misc_feature (1)..(25): Dual Labeled probe 2 SARS-COV2-REV Primer 2
tgctatgtttagtgttccagttttc

SEQ ID NO: 22 - DNA, 25 bp, Artificial Sequence
  Dual Labeled probe 2
  misc_feature (1)..(25): Dual Labeled probe 2
aaggatcagtgccaagctcgtcgcc

SEQ ID NO: 23 - DNA, 25 bp, Artificial Sequence
  Dual Labeled probe 3 SARS-COV2-FOR Primer 3
  misc_feature (1)..(25): Dual Labeled probe 3 SARS-COV2-FOR Primer 3
caggtatatgcgctagttatcagac

SEQ ID NO: 24 - DNA, 24 bp, Artificial Sequence
  Dual Labeled probe 3 SARS-COV2-REV Primer 3
  misc_feature (1)..(24): Dual Labeled probe 3 SARS-COV2-REV Primer 3
ccaagtgacatagtgtaggcaatg

SEQ ID NO: 25 - DNA, 25 bp, Artificial Sequence
  Dual Labeled probe 3
  misc_feature (1)..(25): Dual Labeled probe 3
agactaattctcctcggcgggcacg

SEQ ID NO: 26 - DNA, 24 bp, Artificial Sequence
  Dual Labeled probe 4 SARS-COV2-FOR Primer 4
  misc_feature (1)..(24): Dual Labeled probe 4 SARS-COV2-FOR Primer 4
gcaggtatatgcgctagttatcag

SEQ ID NO: 27 - DNA, 25 bp, Artificial Sequence
  Dual Labeled probe 4 SARS-COV2-REV Primer 4
  misc_feature (1)..(25): Dual Labeled probe 4 SARS-COV2-REV Primer 4
acactggtagaatttctgtggtaac

SEQ ID NO: 28 - DNA, 25 bp, Artificial Sequence
  Dual Labeled probe 4
  misc_feature (1)..(25): Dual Labeled probe 4
agactaattctcctcggcgggcacg

SEQ ID NO: 29 - DNA, 27 bp, Artificial Sequence
  Influenza A virus-FOR Primer 1
  misc_feature (1)..(27): Influenza A virus-FOR Primer 1
atgagycttytaaccgaggtcgaaacg

SEQ ID NO: 30 - DNA, 23 bp, Artificial Sequence
  Influenza A virus-REV Primer 1
  misc_feature (1)..(23): Influenza A virus-REV Primer 1
tggacaaancgtctacgctgcag

SEQ ID NO: 31 - DNA, 22 bp, Artificial Sequence
  Influenza A virus-FOR Primer 2
  misc_feature (1)..(22): Influenza A virus-FOR Primer 2
gagctcagtgtcatcatttgaa

SEQ ID NO: 32 - DNA, 20 bp, Artificial Sequence
  Influenza A virus-REV Primer 2
  misc_feature (1)..(20): Influenza A virus-REV Primer 2
tgctgagctttgggtatgaa

SEQ ID NO: 33 - DNA, 23 bp, Artificial Sequence
  Influenza A virus-FOR Primer 3
  misc_feature (1)..(23): Influenza A virus-FOR Primer 3
cagggaatggtagatggttggta

SEQ ID NO: 34 - DNA, 22 bp, Artificial Sequence
  Influenza A virus-REV Primer 3
  misc_feature (1)..(22): Influenza A virus-REV Primer 3
cggcctcaaactgagtrttcat

SEQ ID NO: 35 - DNA, 23 bp, Artificial Sequence
  Influenza A virus-FOR Primer 4
  misc_feature (1)..(23): Influenza A virus-FOR Primer 4
gtgacgaattcatcaatgtrccg

SEQ ID NO: 36 - DNA, 25 bp, Artificial Sequence
  Influenza A virus-REV Primer 4
  misc_feature (1)..(25): Influenza A virus-REV Primer 4
ctctggtttagtgttgatgtyccaa

SEQ ID NO: 37 - DNA, 22 bp, Artificial Sequence
  Influenza B virus-FOR Primer 1
  misc_feature (1)..(22): Influenza B virus-FOR Primer 1
acataccctcggcaagagtttc

SEQ ID NO: 38 - DNA, 23 bp, Artificial Sequence
  Influenza B virus-REV Primer 1
  misc_feature (1)..(23): Influenza B virus-REV Primer 1
tgctgttttgttgttgtcgtttt

SEQ ID NO: 39 - DNA, 21 bp, Artificial Sequence
  Influenza B virus-FOR Primer 2
  misc_feature (1)..(21): Influenza B virus-FOR Primer 2
acaccttctgcgaaagcttca

SEQ ID NO: 40 - DNA, 24 bp, Artificial Sequence
  Influenza B virus-REV Primer 2
  misc_feature (1)..(24): Influenza B virus-REV Primer 2
catagaggttcttcatttgggttt

(19) United States
(12) Patent Application Publication
(10) Pub. No.: US 2022/0333215 A1
(43) Pub. Date: Oct. 20, 2022

(54) METHODS AND COMPOSITIONS FOR DETECTING CO-INFECTION WITH SARS-COV-2 AND INFLUENZA A VIRUS AND/OR INFLUENZA B VIRUS

(71) Applicants: Yang Xu, Caldwell, NJ (US); Xi Xu, Chicago, IL (US)

(72) Inventors: Yang Xu, Caldwell, NJ (US); Xi Xu, Chicago, IL (US)

(73) Assignee: THE HOPES INSTITUTE, FAIRFIELD, NJ (US)

(21) Appl. No.: 17/218,930

(22) Filed: Mar. 31, 2021

Related U.S. Application Data
(60) Provisional application No. 63/003,079, filed on Mar. 31, 2020.

(51) Int. Cl. C12Q 1/70

(52) U.S. Cl. CPC C12Q 1/701

(57) ABSTRACT

This invention relates generally to the field of virus detection. In particular, the invention provides kits, probes, primers, kits and methods for amplifying and detecting sequences selected from the group comprising COIN-CoV2-241, COIN-CoV2-3037, COIN-CoV2-14408, COIN-CoV2-28144, COIN-CoV2-23403, COIN-CoV2-28881, COIN-CoV2-28882, COIN-CoV2-28883, COIN-CoV2-17747, COIN-IFA-238 and COIN-IFB-002 in samples from co-infection of SARS-CoV-2, influenza A virus (IFA) and/or influenza B virus (IFB). The clinical and other uses of the present kits, probes, primers, kits and methods are also contemplated.

### OBJECT OF THE INVENTION

The object of the invention is a kit for detecting a cause of co-infection with severe acute respiratory syndrome coronavirus 2 (SARS-CoV-2) and non-biological SARS-CoV-2 infection, such as influenza A virus (IFA) or influenza B virus (IFB) and their broad application in diagnostics of co-infection aroused by SARS-CoV-2 or IFA or IFB in humans.

### SEQUENCE LISTING

The instant application contains a Sequence Listing which has been submitted electronically in ASCII format and is hereby incorporated by reference in its entirety. Said ASCII copy, created on Nov. 8, 2021, is named TUP66355 SEQ LIST_ST25 and is 16,384 bytes in size.

### BACKGROUND OF THE INVENTION

The outbreak of severe acute respiratory syndrome coronavirus 2 (SARS-CoV-2) at the end of 2019 has led to a worldwide pandemic. As of Jan. 13, 2021, there has been more than 90 million confirmed infection cases and 1.9 million deaths globally (https://covid19.who.int/). The ending time and the final severity of the current COVID-19 pandemic wave is still uncertain. Meanwhile, the influenza season is merging with the current pandemic, potentially bringing more challenges and posing a larger threat to public health. There are many debates on whether the seasonal flu will impact the severity of the COVID-19 pandemic and whether COVID-19/influenza co-infection investigation is necessary for the next several winters.

The first COVID-19 patient studied was a 41-year-old man with no history of hepatitis, tuberculosis or diabetes. He was admitted to and hospitalized in the Central Hospital of Wuhan on Dec. 26, 2019, 6 days after the onset of disease. The patient reported fever, chest tightness, unproductive cough, pain and weakness for 1 week on presentation. Physical examination of cardiovascular, abdominal and neurological characteristics was that these were normal. Mild lymphopoenia (defined as less than 9×10⁵ cells per ml) was observed, but white blood cell and blood platelet counts were normal in a complete blood count test. Elevated levels of C-reactive protein (41.4 mg 1⁻¹ of blood; reference range, 0-6 mg 1⁻¹) were observed and the levels of aspartate aminotransferase, lactic dehydrogenase and creatine kinase were slightly elevated in blood chemistry tests. The patient had mild hypoxemia with oxygen levels of 67 mm Hg as determined by an arterial blood gas test. On the first day of admission (day 6 after the onset of disease), chest radiographs were abnormal with air-space shadowing such as ground-glass opacities, focal consolidation and patchy consolidation in both lungs. Computed-tomography scans of the chest revealed bilateral focal consolidation, lobar consolidation and patchy consolidation, especially in the lower lung. A chest radiograph revealed a bilateral diffuse patchy and fuzzy shadow on day 5 after admission (day 11 after the onset of disease). Although a combination of antibiotic, antiviral and glucocorticoid therapy was administered, the patient exhibited respiratory failure and was given high-flow non-invasive ventilation. The condition of the patient did not improve after 3 days of treatment and he was admitted to the intensive care unit. The patient was transferred to another hospital in Wuhan for further treatment 6 days after admission.

DEEP META-TRANSCRIPTOMIC SEQUENCING WAS PERFORMED ON SAMPLES FROM LYMPHOCTES. The genome sequence of this virus, as well as its termini, was determined and confirmed by reverse-transcription PCR (RT-PCR) and 5′/3′ rapid amplification of cDNA ends (RACE).

SARS-CoV-2 has a genetic proofreading mechanism achieved by non-structure protein (NSP) 14 in synergy with NSP10 and NSP12. Therefore, SARS-CoV-2 has a higher fidelity in its transcription and replication process than that of other single stranded RNA viruses. Nonetheless, 7123 single mutations have been detected in the 12,754 US SARS-CoV-2 strains in the past with respect to the first SARS-CoV-2 genome collected on Dec. 24, 2019. Genome sequencing, SNP calling, and phenotyping supply an efficient means to study the epidemiology of COVID-19 and infer the relationship between SARS-CoV-2 protein structures and COVID-19 pathogenicity. Analyzing genome sequencing and single-nucleotide polymorphism (SNP) calling has been a hotspot for a wide variety of epidemiological, clinical, experimental, biophysical, mathematical, and computational studies.

Total RNA was extracted from 200 μL of samples and a meta-transcriptomic library was constructed for pair-end (150-bp reads) sequencing using an Illumina MiniSeq. The genome sequence of the virus, as well as its termini, were determined and confirmed by reverse-transcription PCR (RT-PCR) and 5′/3′ rapid amplification of cDNA ends (RACE). This virus whole genome sequence and SNP have been found as SARS-CoV-2, IFA or IFB mutation sub-strains compared to GenBank. Based on the analysis of all mutations, it was uncovered that there are novel SARS-CoV-2, IFA or IFB sub-strains with mutation in samples of lymphocytes (Table 1).

The flu season with the current COVID-19 pandemic raises a potentially severe threat to public health. Through experimental co-infection with IFA and either pseudotyped or live SARS-CoV-2 virus, researchers found that IFA pre-infection significantly promoted the infectivity of SARS-CoV-2 in a broad range of cell types. Remarkably, in vivo, increased SARS-CoV-2 viral load and more severe lung damage were observed in mice co-infected with IFA (Bai L, et al. Cell Research (2021) 0:1-9; https://doi.org/10.1038/s41422-021-00473-1).

WHO Director-General declared COVID-19 pandemic on Mar. 11, 2020 and President of the United States announced a ban on travel from European countries to the United States.

If person is infected with IFA first and then is co-infection with SARS-CoV-2; or a person is infected with SARS-CoV-2 first and then is co-infection with IFA, this may be one of the most possible cause of pandemic!

If IFA and any deadly virus co-infected to people and make a large number of patients go to the hospital for help, in turn, a large number of clinicians and nurses are infected by both IFA and a deadly virus (e.g. SARS-CoV and SARS-CoV-2). The hospital out of control might be the main cause occurred in influenza pandemics of recent 100 years.

From FIG. 1, it indicated that influenza A virus began to infect people in December, gradually increasing the proportion of infected people. Therefore, the co-infection with SARS-CoV-2 will mainly be influenza A virus in January-March. US CDC estimates that, from Oct. 1, 2019, through Mar. 21, 2020, there have been 38,000,000-54,000,000 flu illnesses. Therefore, the major co-infection with SARS-CoV-2 may be influenza B virus or influenza A virus based on prediction from US CDC (FIG. 1).

US CDC issued a warning of co-circulation of influenza viruses and SARS-CoV-2 on Sep. 17, 2020.

During periods of community co-circulation of influenza viruses and SARS-CoV-2, empiric antiviral treatment of influenza is recommended as soon as possible for the following priority groups: a) hospitalized patients with respiratory illness; b) outpatients with severe, complicated, or progressive respiratory illness; and c) outpatients at higher risk for influenza complications who present with any acute respiratory illness symptoms (with or without fever).

Influenza and COVID-19 have overlapping signs and symptoms. Testing can help distinguish between influenza virus infection and SARS-CoV-2 infection.

Co-infection IFA or IFB with SARS-CoV-2 can occur and should be considered, particularly in hospitalized patients with severe respiratory disease. For hospitalized patients with suspected influenza who are started on empiric antiviral treatment with oseltamivir, use of influenza molecular assays or multiplex assays that detect both influenza viruses and SARS-CoV-2 can inform clinical management.

Patients at higher risk for influenza complications should be advised to call their provider as soon as possible if they have acute respiratory illness symptoms (with or without fever) for consideration of infection with IFA or IFB (and early antiviral treatment), SARS-CoV-2, and other respiratory pathogens.

Clinical algorithms for the testing and treatment of influenza when SARS-CoV-2 and influenza viruses are circulating are also available.

The golden standard test will be nuclear acid test (RT-PCR) for SARS-CoV-2 and influenza A virus (IFA) and/or influenza B virus (IFB). For patients who develop a productive cough, sputum should be collected and tested for SARS-CoV-2 and IFA and/or IFB.

If a patient was diagnosed COVID-19 and the patient appearing phenomenon of lymphocyte depletion (PLD) in hospital. This is an indication of co-infection with IFA or IFB. An immunoassay or RT-PCR for IFA or IFB may be conducted.

SARS-CoV-2 present detecting method of three types: immunoassay (e.g. ELISA), reverse transcriptase polymerase chain reaction (RT-PCR) tests and cell culture.

The three detecting methods have significant drawbacks. For example, ELISA can be reliably detected antibodies from the serum of COVID-19 patient. However, these antibodies were detected in only 7-21 days after symptoms occur. The cell culture method of the detection period is relatively long, and is applicable only to a limited condition. In addition, cell culture methods can only detect the presence of the viable virus.

The key steps to prevent the spread of SARS-CoV-2 are early diagnosis, early isolation and treatment. RT-PCR is the only way to detect SARS-CoV-2 nucleic acid present. However, SARS-CoV-2 RT-PCR cannot rule out the co-infected patient with IFA or IFB. Therefore, SARS-CoV-2 RT-PCR cannot meet the needs of early clinical screening and diagnosis of co-infection SARS-CoV-2 with IFA and/or IFB. COVID-19 diagnostic techniques require a rapid, sensitive and accurate in the art of COVID-19 management. The present invention addresses this need in the art and other related needs. We have developed a real-time PCR-based mutation assay to detect single point substitutions, insertions, or deletions for co-infection SARS-CoV-2 with IFA and/or IFB.

### BRIEF DESCRIPTION OF THE DRAWINGS

The above and other objectives and advantages of the present invention will be more fully understood with reference to the following detailed descriptions, taken in conjunction with the accompanying drawings, in which:

FIG. 1 is a prediction influenza B virus or influenza A virus infection from US CDC.

FIG. 2 is an SNP MALDI analysis.

FIG. 3 is a diagram to illustrate the invention method.

FIG. 4 is a comparison of length of influenza A and B virus proteins.

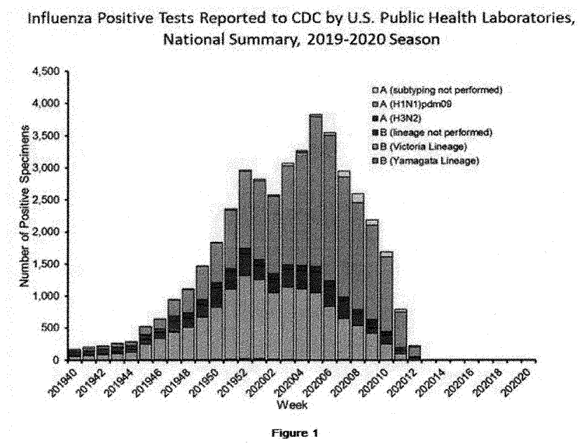

Supply Sample 1. Features of COVID-19 patients (lymphopenia recovery after 2 weeks).

Supply Sample 2. Features of COVID-19 patients co-infection with IFA (influenza A virus).

Supply Sample 3. Features of lymphocyte infection in COVID-19 patients co-infected with IFA (influenza A virus).

Supply Sample 4. Evidence of the key features of COVID-19 co-infection with influenza A virus (IFA).

Supply Sample 5. Double-blind test co-infection influenza A or B virus in COVID-19 patients.

Supply Sample 6. Comparison of influenza virus and SARS-CoV2 properties and treatments.

Table 1. Sequence listing of gene mutations identified from lymphocytes of co-infection with SARS-CoV2, IVA or IVB represented by SEQ ID NO: 1 with the mutation C241A, 5′UTR referred to as COIN-CoV2-241; SEQ ID NO: 2 with mutation C3037T, ORF1ab referred to as COIN-CoV2-3037; SEQ ID NO: 3 with mutation C14408A, ORF1ab referred to as COIN-CoV2-14408; SEQ ID NO: 4 with mutation T28144C, ORF8 referred to as COIN-CoV2-28144; SEQ ID NO: 5 with mutation A23403C, Protein S referred to as COIN-CoV2-23403; SEQ ID NO: 6 with mutation G28881A, Protein N referred to as COIN-CoV2-28881; SEQ ID NO: 7 with mutation G28882T, Protein N referred to as COIN-CoV2-28882; SEQ ID NO: 8 with mutation G28883A, Protein N referred to as COIN-CoV2-28883; SEQ ID NO: 9 with mutation C17747T, Protein N SP 13 referred to as COIN-CoV2-17747; SEQ ID NO: 10 with mutation C238A, ORF referred to as COIN-IFA-238; SEQ ID NO: 11 with an influenza B viruses (“B/Victoria/2/87-like”) mutation at hemagglutinin (HA) protein at D129G; and SEQ ID NO: 12 with an influenza B viruses (“B/Victoria/2/87-like”) mutation at hemagglutinin (HA) protein at R498K, which may occur individually or simultaneously and are referred to as COIN-IFB-002.

Table 2. Exemplary human SARS-CoV-2 primer, where SYBR Green SARS-COV2-FOR Primer 1 or SEQ ID NO: 13 is the first forward primer; SYBR Green SARS-COV2-REV Primer 1 or SEQ ID NO: 14 is the first reverse primer; SYBR Green SARS-COV2-FOR Primer 2 or SEQ ID NO: 15 is the second forward primer; SYBR Green SARS-COV2-REV Primer 2 or SEQ ID NO: 16 is the second reverse primer; Dual Labeled probe 1 SARS-COV2-FOR Primer 1 or SEQ ID NO: 17 is the first dual labeled forward primer; Dual Labeled probe 1 SARS-COV2-REV Primer 1 or SEQ ID NO: 18 is the first dual labeled reverse primer; Dual labeled probe 1 or SEQ ID NO: 19 is the first dual labeled probe; Dual Labeled probe 2 SARS-COV2-FOR Primer 2 or SEQ ID NO: 20 is the second dual labeled forward primer; Dual Labeled probe 2 SARS-COV2-REV Primer 2 or SEQ ID NO: 21 is the second dual labeled reverse primer; Dual labeled probe 2 or SEQ ID NO: 22 is the second dual labeled probe; Dual Labeled probe 3 SARS-COV2-FOR Primer 3 or SEQ ID NO: 23 is the third dual labeled forward primer; Dual Labeled probe 3 SARS-COV2-REV Primer 3 or SEQ ID NO: 24 is the third dual labeled reverse primer; Dual labeled probe 3 or SEQ ID NO: 25 is the third dual labeled probe; Dual Labeled probe 4 SARS-COV2-FOR Primer 4 or SEQ ID NO: 26 is the fourth dual labeled forward primer; Dual Labeled probe 4 SARS-COV2-REV Primer 4 or SEQ ID NO: 27 is the fourth dual labeled reverse primer; Dual labeled probe 4 or SEQ ID NO: 28 is the fourth dual labeled probe.

Table 3. Exemplary influenza A virus primer, where SEQ ID NO: 29 is Influenza A virus-FOR Primer 1 or first forward primer; SEQ ID NO: 30 is Influenza A virus-REV Primer 1 or first reverse primer; SEQ ID NO: 31 is Influenza A virus-FOR Primer 2 or second forward primer; SEQ ID NO: 32 is Influenza A virus-REV Primer 2 or second reverse primer; SEQ ID NO: 33 is Influenza A virus-FOR Primer 3 or third forward primer; SEQ ID NO: 34 is Influenza A virus-REV Primer 3 or third reverse primer; SEQ ID NO: 35 is Influenza A virus-FOR Primer 4 or fourth forward primer; and SEQ ID NO: 36 is Influenza A virus-REV Primer 4 or fourth reverse primer.

Table 4. Exemplary influenza B virus primer, where SEQ ID NO: 37 is Influenza B virus-FOR Primer 1 or first forward primer; SEQ ID NO: 38 is Influenza B virus-REV Primer 1 or first reverse primer; SEQ ID NO: 39 is Influenza B virus-FOR Primer 2 or second forward primer; SEQ ID NO: 40 is Influenza B virus-REV Primer 2 or second reverse primer.

### DISCLOSURE OF INVENTION

Sequences of SEQ ID NO: 1 referred to as COIN-CoV2-241 with a mutation of C241A residue in the 5′UTR region of SARS-CoV2, SEQ ID NO: 2 referred to as COIN-CoV2-3037 with a mutation of C3037T residue in the ORF1ab of SARS-CoV-2, SEQ ID NO: 3 referred to as COIN-CoV2-14408 with a mutation of C14408A residue in the ORF1ab of SARS-CoV2, SEQ ID NO: 4 referred to as COIN-CoV2-28144 with a mutation of T28144C in the ORF8 of SARS-CoV-2, SEQ ID NO: 5 referred to as COIN-CoV2-23403 with a mutation of A23403C in the Protein S of SARS-CoV2, SEQ ID NO: 6 referred to as COIN-CoV2-28881 with a mutation of G28881A in the Protein N of SARS-CoV-2, SEQ ID NO: 7 referred to as COIN-CoV2-28882 with a mutation of G28882T in the Protein N of SARS-CoV-2, SEQ ID NO. 8 referred to as COIN-CoV2-28883 with a mutation of G28883A in the Protein N of SARS-CoV-2, SEQ ID NO: 9 referred to as COIN-CoV2-17747 with a mutation of C17747T in the NSP13 of SARS-CoV-2, SEQ ID NO: 10 referred to as COIN-IFA-238 with a mutation of C238A residue in ORF of Influenza A virus H1N1, and SEQ ID NO: 11 and/or SEQ ID NO: 12 collectively referred to as COIN-IFB-002 with an influenza B viruses (“B/Victoria/2/87-like”) mutation at hemagglutinin (HA) protein at D129G or R498K as also listed in Table 1.

This invention relates generally to the field of virus detection. In particular, the invention provides kits, probes, primers, kits and methods for amplifying and detecting co-infection of SARS-CoV-2 with influenza A virus (IFA) and/or influenza B virus (IFB). The clinical and other uses of the present kits, probes, primers, kits and methods are also contemplated.

Current clinical data suggest that many suspected cases of COVID-19 are not actually infected with the SARS-CoV-2, on the contrary, infected with other pathogens. Therefore, development of a method to simultaneously detect SARS-CoV-2 and other pathogens cause similar symptoms of COVID-19. Such an approach would provide rapid screening of suspected cases in order to reduce the likelihood of diagnostic error, allowing timely and appropriate treatment and avoid unnecessary panic and medical waste. Patients infected with the SARS-CoV-2 more susceptible to other pathogens infection, which is due to the SARS-CoV-2 that causes decreased immunity. COVID-19 patients can be infected with SARS-CoV-2 and other pathogens produce similar symptoms. For example, if a patient infected with both COVID-19 also infected with IFA, then use only drug treatment for COVID-19 will not be able to make symptoms disappear immediately. In this case, the simultaneous detection of two pathogens the infection would allow patients infected with both pathogens and effective treatment immediately. Based diagnostic Bio-kit is a high-throughput screening of multiple samples simultaneously for a fast and low-cost method. It is therefore an object of the present invention is to provide a Bio-kit, for simultaneously detecting SARS-CoV-2 and other pathogens, for example IFA or IFB, cause a COVID-19-like symptoms.

In one aspect, the present invention relates to a kit for detecting the cause of SARS-CoV-2, IFA, and/or IFB infection, the kit comprising a support, suitable for use in nucleic acid hybridization, the oligonucleotide probe is fixed to a nucleotide sequence of the genome of SARS-CoV-2 is complementary to the support, the nucleotide sequence comprises at least 10 nucleotides, and comprising one or more of the following oligonucleotide acid probe: a) a SARS-CoV-2 symptoms and leads to a SARS-CoV-2 infection organism nucleotide sequence complementary to an oligonucleotide probe, the nucleotide sequence comprises at least 10 nucleotides; or b) a non-SARS-CoV-2, IFA or IFB, a nucleotide sequence complementary to the viral oligonucleotide probe, the nucleotide sequence comprises at least 10 nucleotides.

In some embodiments, a kit of the present invention comprises a support, suitable for use in nucleic acid hybridization, the support may be fixed with at least two different nucleotide sequences of the SARS-CoV-2 genome at least two complementary oligonucleotide nucleotide probe, a nucleotide sequence of each of the two different nucleotide sequences comprising at least 10 nucleotides.

Another aspect, the present invention relates to a method for detection of SARS-CoV-2 in the sample and non-biological SARS-CoV-2 infection, the method comprising: a) providing one of the above described kit; b) containing or suspected of containing SARS-CoV-2 samples and non-biological SARS-CoV-2 infection of a nucleotide sequence of the kit contacts under conditions suitable for nucleic acid hybridization; and c) evaluating the SARS-CoV-2 or the non-SARS-CoV-2 infection organism nucleotides sequences, if present in the sample, then, and the nucleotide sequence of the SARS-CoV-2 genome complementary to the nucleotide sequence of oligonucleotide probe or the non-SARS-CoV-2 infection in the genome of complementary hybridization complexes formed between the nucleotide probe oligonucleotide, so long as the detection of one or both of the hybridization complex can indicate the presence of the SARS-CoV-2 and/or in said sample non-SARS-CoV-2 infection organisms.

In some embodiments, detection of SARS-CoV-2 by the steps of: a) providing a kit comprising a support material, said kit suitable for use in nucleic acid hybridization, which can be fixed with the SARS-CoV-2 genome of at least two different nucleotide sequence complementary to at least two oligonucleotide probes, each of said two different nucleotide sequences in the nucleotide sequence comprising at least 10 nucleotides; b) containing or suspected of containing SARS-CoV-2 contacts the sample kit nucleotide sequence under conditions suitable for nucleic acid hybridization; and c) evaluating the SARS-CoV-2 nucleotide sequence (if present in the sample), and said at least two hybridization complexes formed between two different nucleotide sequences of the SARS-CoV-2 genome are complementary to oligonucleotide probes to determine the presence and quantity of the sample of SARS-CoV-2, sufficient to detect to one or both of the hybridization complex indicates the presence on the SARS-CoV-2 in the sample.

By using multiple hybridization probes, as compared with a single hybridization probe based assay, the present method reduces the incidence of false negative results, the probability of this is due to the abrupt change in the plurality of targets hybridized at the same time well below the target hybridizing single the probability of emergence of mutations. When other preferred embodiments on the kit, for example, a negative control probe and gaps may reduce the chances of false positive results. On the kit include more preferred embodiment, for example, a control probe and an immobilized probe is a positive control, can provide further validation of the detection results. Using the preferred sample preparation process, RNA extraction procedure and amplification procedures can be further improved sensitivity of the method of the present invention.

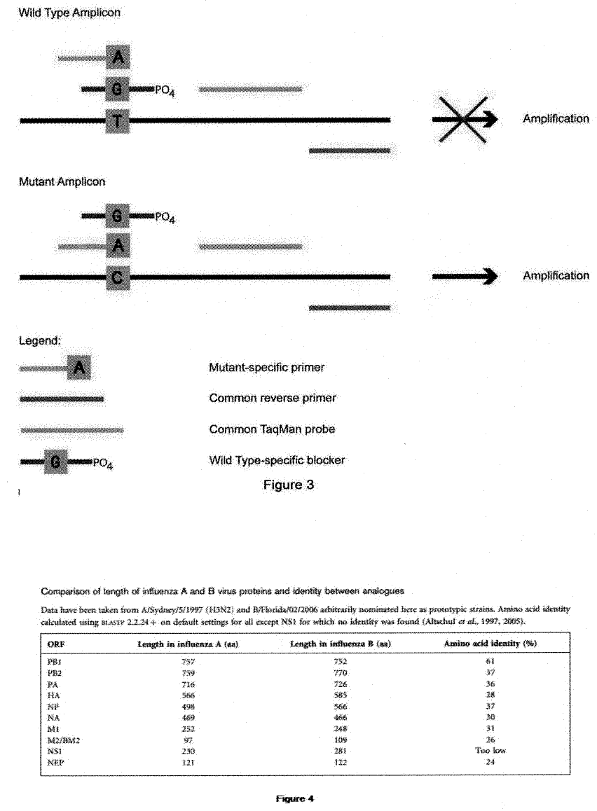

In still another aspect, the present invention relates to a kit for the amplification of IFA or IFB, the kit comprises: a) a primer as described in FIG. 3-4; and b) using the primers of IFA or IFB nucleotide sequence of a nucleic acid polymerase.

In still another aspect, the present invention relates to a oligonucleotide probe, for IFA or IFB nucleoside acid sequence hybridization, the oligonucleotide probe comprises a nucleotide sequence having the following characteristics: a) under high stringency conditions, and the IFA or IFB or a complementary strand; or b) and IFA or IFB target nucleotide sequence or its complementary strand having at least 90% identity.

In still another aspect, the present invention relates to a kit for IFA or IFB, the kit comprising: a) of the probe above; and b) a process, method for evaluating the IFA or IFB nucleotide sequence of the probe between.

Schematically illustrate the exemplary structure of SARS-CoV-2 genome (see Zhu N et al. A novel coronavirus from patients with pneumonia in China, N Engl J Med. 2020. doi:10.1056/NEJMoa2001017.2).

Common sequence of the specific primers is complementary to a universal primer. Universal primers and specific primers are added to the system prior to PCR amplification, guaranteed by specific amplification of a specific portion of the specific primers. After one or a few thermal cycles, universal primers may be efficiently integrated into the amplicon them. Then universal primers can anneal to complementary sequences consensus sequence specific primers.

Amine coupling chemistry: the covalent amine substrate comprising primary amine groups on (NH3+) is connected to the glass surface on. Amine is positively charged at neutral pH, and thus through the negatively charged phosphate backbone to form an ionic bond with negatively charged to attract DNA. By electrostatic adsorption with ultraviolet light irradiation or heating supplement, which in conjunction with by a covalent linkage between the primary amine and thymine so that the DNA can be covalently attached to the surface. Electrostatic binding and covalently linked combination of a highly stable manner in the end was coupled to the DNA.

### EXAMPLE

In order to make clear disclosure, but not limited to, the detailed description of the invention is divided into the following sections.

### Definitions

Unless defined otherwise, all technical and scientific terms used herein have the same meaning as in the art to which this invention belongs commonly understood by one of ordinary skill. All patents referred to herein, an application, and other applications have been disclosed are incorporated herein by reference in its entirety. If this portion is defined according to the patents incorporated herein by reference, the definition of an application, and other applications have been disclosed in the opposite or inconsistent, then the definition of this section are incorporated herein beyond it is defined as a reference.

As used herein, “an” means “at least one” or “one or more.”

As used herein, “coronavirus family” means a respiratory disease single-stranded RNA virus family. Arranged radially outwardly of the rod-like protrusions on the outer membrane of the virus, the virion negative staining shows a typical appearance of the crown.

As used herein, “polymerase chain reaction (the PCR)” refers to in vitro DNA amplification system. In the presence of excess deoxynucleotides and a thermostable DNA polymerase such as Taq DNA polymerase case, two synthetic oligonucleotide primers are added to the target DNA (used without purification), the two synthetic oligonucleotides nucleotide primer with the amplified target DNA (one for each strand) complementary to a region of the two. In a series of temperature cycles, for example, 30 temperature cycles, the target DNA is repeatedly denatured (e.g., about 90 deg.] C.), annealed to the primers (e.g. at 50-60 deg.] C.) and primers from amplified progeny strand (72 deg.] C. e.g.). As the chain itself as the subsequent progeny cycle template, and two primers is amplified DNA fragment matched index, rather than linear amplification. Thus, the original DNA is purified neither need nor require high abundance, PCR reactions are therefore not only in research but also in clinical diagnosis and forensic science have been widely used.

As used herein, a “nested PCR” refers to PCR in which improved specificity by sequentially using two sets of primers. Initial PCR using the “outer” primer pair, and an aliquot of the first PCR product was used as template in a second round of PCR, the second round of PCR using the “inner” primer pair.

As used herein, “reverse transcription PCR or RT-PCR” refers to PCR, in which the initial template is RNA, which means an initial step of reverse transcriptase to generate DNA templates. Some thermostable polymerases have significant reverse transcriptase activity, however, more often it is a significant step of reverse transcription, so that inactivation of the reverse transcriptase or the purified product, ultimately a single conventional PCR.

As used herein, “primer” refers to a section of an oligonucleotide, capable of hybridizing with the target sequence, typically during amplification of nucleic acid amplification initiator.

As used herein, “probe” refers to a section of an oligonucleotide, capable of hybridizing with the target sequence, typically to assist in the detection of the target sequence. The term “target sequence” refers to a period of a nucleic acid sequence probe specifically binds. And initiate the amplification program for different target nucleic acid primers, probes need not be extended using a polymerase to amplify the target sequence. However, to those of ordinary skill in the art it will be apparent that the probes and primers are structurally similar in many cases or identical.

As used herein, “the 5′ and 3′ universal primer concentrations are equal to or higher than the 5′ and 3′ specific primers concentration” refers to a 5′ universal primer concentration is equal to or greater than 5′ concentration of the specific primers, and a 3′ universal primer concentration is equal to or higher than 3′ specific primer concentrations.

As used herein, a “hairpin structure” refers to a polynucleotide or nucleic acid comprises a double-stranded stem and a single-stranded loop portion, wherein the two polynucleotides or nucleic acid strand to form a double-stranded stem portion connected together and isolated polynucleotide or nucleic acid strand form a ring by a single portion. “Hairpin structure” may further comprise a single strand from a double stranded stem portion extending out of the 3′ and/or 5′.

As used herein, “nucleic acid” refers to any form of deoxyribonucleic acid (DNA) and/or ribonucleic acid (an RNA), which comprises a single-stranded, double-stranded, triple-stranded, linear and circular forms. Also includes polynucleotides, oligonucleotides, chimeras of nucleic acids and the like. Nucleic acids described herein may be performed by well-known composition of DNA and RNA, the DNA and RNA nucleotide adenosine, cytosine, guanine, thymidine and uridine composition, or may be made of these bases analogs or derivatives thereof. Further, having a plurality of non-traditional diester phosphate backbone oligonucleotides other derivatives are also included therein, such as phosphoric acid triesters, polynucleopeptides (PNA), methylphosphonate, phosphorothioate, polynucleotide primer, blocking nucleic acids (LNA) and the like.

As used herein, “complementary or matched” means that two nucleic acid sequences having at least 50% sequence identity. Preferably, the two nucleic acid sequences having at least 60%, 70%, 80%, 90%, 95%, 96%, 97%, 98%, 99% or 100% sequence identity. “Complementary or matched” also refers to two nucleic acid sequences may be low, the hybridization and/or high stringency conditions.

As used herein, “substantially complementary or substantially matched” means that two nucleic acid sequences having at least 90% sequence identity. Preferably, the two nucleic acid sequences having at least 95%, 96%, 97%, 98%, 99% or 100% sequence identity. Optionally, the “substantially complementary or substantially matched” means that two nucleic acid sequences can hybridize under high stringency conditions.

As used herein, “nucleotide sequence of two accurately matched” refers to a nucleic acid duplex, wherein the two nucleotide chains according to Watson-Crick base pairing rules pairing (Watson-Crick base-pair principle), i.e. DNA:DNA duplex pair CG and the AT, and DNA:RNA or RNA:RNA duplex pair CG and the AU, and are not deleted or inserted in each strand of double-stranded.

As used herein, “stringency of hybridization” with a percentage error as follows: 1) high stringency: 0.1×SSPE (or 0.1×SSC), 0.1% SDS, 65° C.; 2) medium stringency: 0.2×SSPE (or 1.0×SSC), 0.1°% SDS, 50° C. (also referred to as moderate stringency); and low stringency: 1.0×SSPE (or 5.0×SSC), 0.1% SDS, 50° C.

It should be understood that other buffers, salts and temperatures to get the same stringency.

As used herein, “gene” refers to a unit of inheritance occupies a specific site in the chromosome, the presence of the gene may be determined by the appearance of different types of alleles. If given occurrence splicing genes, including a need to produce a single polypeptide of the genomic DNA sequence (exon).

As used herein, “melting temperature” (“Tm”) refers to a nucleic acid duplex i.e. DNA:DNA is denatured temperature range such as: DNA, DNA:RNA, RNA:RNA, PNA:DNA, LNA:RNA and LNA midpoint.

As used herein, “sample” refers to any material that might contain a target amplification or detection of SARS-CoV-2 by kit, primers, probes, methods and kits of the present invention. The sample can be a biological sample, such as a biological fluid (biological fluid) or biological tissue. Examples of biological fluids include urine, blood, plasma, serum, saliva, semen, stool, sputum, cerebral spinal fluid, tears, mucus, amniotic fluid or the like. Biological tissue is a cell population, typically a specific type of cell to their intracellular material polymers, they form one of the structural material of human, animal, plant, bacterial, fungal or viral structure, including connective, epithelial tissue, muscle tissue and nerve tissue. Examples of biological tissues include organs, tumors, lymph nodes, arteries and individual cells. Biological tissues may be processed to get cell suspension samples. Sample preparation may also be a mixture of cells in vitro. Samples may be cultured suspension cells. In the case where the biological sample, the sample may be the original sample or processed sample is original samples prepared after multiple treatments. For example, various cell separation methods (e.g., magnetically activated cell sorting method) may be used as the target cells isolated or enriched blood from the body fluid sample. Sample used in the present invention include such target cells enriched cell preparation.

As used herein, “liquid (fluid) sample” refers to a liquid or fluid samples manner as naturally occurring, e.g. biological fluid. “Liquid sample” also refers to a sample a non-naturally occurring liquid state, such as a solid or a gas, but is prepared containing the solid or gas sample material liquid, fluid, solution or suspension. For example, liquid sample containing liquid may be comprised, fluid, solution or suspension of biological tissue.

As used herein, a specified amount “Evaluation of PCR product” and/or qualitative determination of the PCR product, and also refers to a get index, ratio, percentages, visual or other value to indicate the level of PCR product. Assessment may be direct or indirect, of course, the chemical species actually detected need not be PCR product itself but may for example be a derivative thereof or some further substance.

Detection of SARS-CoV-2 and non-SARS-CoV-2 infection in the biological kit aspect, the present invention relates to a kit for detecting a cause of SARS-CoV-2 infection and non-biological, the kit comprises a support, suitable for use in nucleic acid hybridization, the oligonucleotide probe can be fixed to a nucleotide sequence complementary to the genome of SARS-CoV-2 on the support, the nucleotide sequence comprises at least 10 nucleotides, and a probe oligonucleotide or more of the following oligonucleotide probes: a) a COVID-like symptoms and leads to a non-SARS-CoV-2 infection organism a nucleotide sequence complementary oligonucleotides acid probe, the nucleotide sequence comprises at least 10 nucleotides; or b) a non-oligonucleotide probe SARS-CoV-2 coronavirus 2 family of viruses nucleotide sequence complementary to, the nucleotide sequence comprises at least 10 nucleotides acid.

In some embodiments, the kit comprising a support, suitable for use in nucleic acid hybridization, the support can be fixed on at least two oligonucleotide probes were at least two different cores thereof with SARS-CoV-2, IFA, and/or IFB genome nucleotide sequence complementary to a nucleotide sequence of each of the two different nucleotide sequences comprising at least 10 nucleotides.

The at least two different nucleotide sequences may be any suitable combination. For example, at least two different nucleotide sequences of the SARS-CoV-2 genome may comprise at least 10 nucleotides of the nucleotide sequence of the genome of SARS-CoV-2 is located in the conserved regions, located SARS-CoV-2 genome variable region at least 10 nucleotides in the nucleotide sequence. In another embodiment, the at least two different nucleotide sequences of the SARS-CoV-2 genome may comprise at least 10 nucleotides of the sequence of nucleotides located in SARS-CoV-2 genome encoding the structural protein gene, located at least 10 nucleotides of the nucleotide sequence of the nonstructural protein-encoding gene in the genome—SARS-CoV-2.

If desired, the kit of the present invention may include a probe or other features of other types. For example, the kit may further comprise: a) three kinds of oligonucleotide probes of at least one of the following oligonucleotide probes: one immobilized control probes, the probe is labeled, and when comprising or suspected of containing SARS-CoV-2 infection in a biological sample in contact with the kit, the probe does not participate in any hybridization reaction; a positive control probe, the probe or any SARS-CoV-2 infection organism sequence is not complementary to, but not found in the sample with SARS-CoV-2 or non-SARS-CoV-2 infection in the organism comprises a sequence complementary to; a negative control probe, the probe and any nucleotide sequence not complementary contained in the sample; and b) a blank spot.

In certain embodiments, the kit of the present invention comprises at least two oligonucleotide probes, two oligonucleotide probes respectively comprising two different nucleotide sequence complementary to at least 10 nucleotides the two different nucleotide sequences comprising at least 10 nucleotides in a conserved region of SARS-CoV-2 genome, a gene encoding the protein in a SARS-CoV-2 genome or in the genome of SARS-CoV-2 a non-structural protein coding gene.

Any conserved region of the genome of SARS-CoV-2 may be used as an experimental target substance. For example, the conserved region of the genome of SARS-CoV-2 may be located in the SARS-CoV-2 replicase. 1A, 1B gene or a region of the nucleocapsid (N) gene.

Any of the variable regions of SARS-CoV-2 genome can be used as an experimental target substance. For example, the variable region of SARS-CoV-2 genome can be located in a region of SARS-CoV-2 spike glycoprotein (S) gene.

Any structural gene encoding the SARS-CoV-2 genome can be used as an experimental target substance. For example, the structural protein genes of SARS-CoV-2 genome encoding may spike glycoprotein (S), the small envelope protein gene (E) or nucleocapsid protein (N).

Any non-structural protein coding gene of SARS-CoV-2 genome may be used to identify a target substance. For example, non-structural protein genes of SARS-CoV-2 genome may be a gene encoding an enzyme replication 1A or 1B.

In another particular embodiment, the kit of the present invention may comprise at least two oligonucleotide probes four oligonucleotide probes of the following: two replication of SARS-CoV-2 enzyme located 1A or 1B at least two different nucleotide sequences complementary to the 10 nucleotides of the gene oligonucleotide probe complementary to a nucleotide sequence of the N genes of SARS-CoV-2 at least 10 nucleotides oligonucleotide probe and a complementary nucleotide sequence at least 10 nucleotides of the SARS-CoV-2 S gene located in the oligonucleotide probes.

Preferably, located in the SARS-CoV-2 replicase or 1 or 1B two different nucleotide sequences in the gene may comprise a nucleotide sequence having the following features: a) at high stringency conditions a nucleotide sequence or a complementary strand replicase 1A or 1B; or b) a nucleotide sequence comprising a replication enzyme in a 1A or 1B having a nucleotide sequence or a complementary strand of at least 90% identity. More preferably, located in the SARS-CoV-2 replicase 1A or one or two different nucleotide sequences as a nucleotide sequence 1B gene.

And the nucleotide sequence preferably, situated in the SARS-CoV-2 N gene may comprise a nucleotide sequence having the following features: a) the nucleotide sequence of N gene under high stringency conditions or its complementary strand; orb) comprises the nucleotide sequence shown in table 13, the N gene or its complementary strand having a nucleotide sequence at least 90% identity.

And the nucleotide sequence preferably, situated in the SARS-CoV-2 S gene may comprise a nucleotide sequence having the following features: a) the nucleotide sequence of the S gene under high stringency conditions or its complementary strand; or b) comprises the nucleotide sequence S gene or its complementary strand having a nucleotide sequence at least 90% identity.

Any suitable label may be used in an immobilized control probe, such as chemical, enzymatic, immunogenic, radioactive, fluorescent, luminescent markers or FRET.

Any suitable non-SARS-CoV-2 sequences may be used. For example, non-SARS-CoV-2 sequences may be endogenous components of the test sample. Optionally, the non-SARS-CoV-2 sequences in a sample are to be detected is enhanced (spiked). Non-SARS-CoV-2 sequences in another embodiment, enhancement may be a sequence of Arabidopsis thaliana (Arabidopsis) origin.

In still another particular embodiment, the present invention may include kit located two of SARS-CoV-2 replicase 1A or 1B two different gene nucleotide sequences complementary to the oligonucleotide probes; a oligonucleotide probes complementary to the SARS-CoV-2 located in the N gene nucleotide sequence; an oligonucleotide probe complementary to a sequence located in the SARS-CoV-2 S gene nucleotide; an immobilization control probes, the probe is labeled, when containing or suspected of containing SARS-CoV-2 or SARS-CoV-2 infection in a biological sample in contact with the kit, the probe does not participate in any hybridization reaction; a positive control probe, which not complementary to any sequence of SARS-CoV-2, but contained in the sample, any sequence not found in the SARS-CoV-2 or SARS-CoV-2 infection in a biological complementary; a negative control probe, which contains any core in the sample nucleotide sequence is not complementary.

Preferably, the kit includes a plurality of probe points as described, for example, a plurality of probe points as follows: two located SARS-CoV-2 replicase genes 1A or 1B of two different nucleotide sequences complementary to oligonucleotide probes, located in the nucleotide sequence of the SARS-CoV-2 N gene oligonucleotide probes complementary to, the nucleotide sequence is located in the SARS-CoV-2 S gene complementary oligonucleotide probe, the immobilized control probe, the positive control probe and negative control probe.

Kit of the present invention may further comprise a nucleotide sequence with an oligonucleotide probe does not involve the SARS-CoV-2 complementary. For example, not related to the SARS-CoV-2 may be I, II or III coronavirus 2 group, or the type of infection following a coronavirus: birds, such as avian infectious bronchitis virus and the avian infectious laryngotracheitis virus; Ma class, such as horses coronavirus; dogs, such as canine coronavirus; cats, such as feline coronavirus and feline infectious peritonitis virus; pig category, such as porcine epidemic diarrhea virus and porcine transmissible gastroenteritis virus, porcine condensate encephalomyelitis virus; calf class, e.g. newborn calf diarrhea coronavirus; bovine, bovine coronavirus; the murine, such as mouse hepatitis virus; finches beak-sea, sea bird beaks e.g. virus; large rodents, such as rats and saliva coronavirus virus pancreatitis rats projects; for example, turkeys class, such as turkey coronavirus; or a human, such as human enteric coronavirus. Kit of the present invention may further comprise an oligonucleotide probe complementary to the nucleotide sequence of other types of virus or pathogen.

A plurality of probes, such as oligonucleotide probes complementary to the SARS-CoV-2 genome is located in conserved regions in the nucleotide sequence, the nucleotide sequence of the variable region of the SARS-CoV-2 genome complementary oligonucleotide nucleotide probe immobilized control probe, the positive control probe or negative control probe, the nucleotide sequence of a SARS-like symptoms lead to non-SARS-CoV-2 infection organism complementary oligonucleotide probes, infection and destruction nucleotide sequence complementary to an oligonucleotide probe host immune system non-SARS-CoV-2 infection organism, nucleotide sequence and a non-SARS-CoV-2 coronavirus family of viruses complementary oligonucleotide probes, in which 5′ end may comprise one poly-dT region in order to enhance its immobilized on a support.

In a particular embodiment, at least a nucleotide sequence complementary is highly expressed SARS-CoV-2 genomes of these oligonucleotide probes. Such kits are particularly useful in the early detection of SARS-CoV-2 infection.

In some embodiments, the non-SARS-CoV-2 infection organism is a biological cause infection COVID-19-like symptoms. Such organisms include, but are not limited to IFA or IFB.

And a target oligonucleotide probe SARS-CoV-2 and the nucleotide sequence of any non SARS-CoV-2 infection organism may be any suitable length. Preferably, the oligonucleotide probes and SARS-CoV-2, and any non-target nucleotide sequence length SARS-CoV-2 infection organism is at least 7, 10, 20, 30, 40, 50, 60, 80, 90, 100 or more than 100 nucleotides.

Oligonucleotide probes and primers may be prepared by any suitable method, for example, chemical synthesis, recombinant methods and/or using both methods (see generally, Ausubel et al., (Eds.), Current Protocols in Molecular Biology, John Wiley & amp; Sons, Inc (2000).

Any suitable support may be used in the kit of the present invention. For example, the support may comprise a material selected from silicon, plastic, glass, ceramics, rubber and the surface of the polymer surface.

In another aspect, the present invention relates to a method for detection of SARS-CoV-2 in the sample and non-SARS-CoV-2 infection organism, the method comprising: a) providing said kit; b) with a sample containing or suspected of containing the nucleotide sequence of SARS-CoV-2 and non-SARS-CoV-2 infection in the organism under conditions suitable for nucleic acid hybridization contacting the kit; and c) evaluating the SARS-CoV-2 or the non-SARS-CoV-2 infection organism a nucleotide sequence (if present in the sample), and the nucleotide sequence of the SARS-CoV-2 genome complementary to said oligonucleotide probe or the hybridization complexes formed between the oligonucleotide probe nucleotide sequence complementary to the non-SARS-CoV-2 infection in the genome of an organism, as long as the detection of one or both of the hybridization complex indicates that it is the presence of SARS-CoV-2 and/or the non-SARS-CoV-2 infection in the biological sample described later.

In some embodiments, the detection of SARS-CoV-2 by the steps of: a) providing a kit that comprises a support, suitable for use in nucleic acid hybridization, the support can be fixed on the object with the SARS-CoV-2 genome at least two different nucleotide sequences complementary to the at least two oligonucleotide probes, each of said two different nucleotide sequences in the nucleotide sequence comprising at least 10 nucleotides; b) containing or a sample suspected of containing SARS-CoV-2 nucleotide sequence under conditions suitable for nucleic acid hybridization contacting the kit; and c) nucleotide sequence of the SARS-CoV-2 evaluation (if present in the sample), and a SARS-CoV-2 hybridization complexes formed between two of the at least two different oligonucleotide probes—CoV genomic nucleotide sequences are complementary to said, to determine the presence in the sample or not, or a SARS-CoV-2 number, so long as the detection of one or both of the hybridization complex indicates the presence on the SARS-CoV-2 in the sample.

In a particular embodiment, the method of the present invention comprises: a) providing a kit, the kit comprising a nucleotide sequence conserved region of SARS-CoV-2 genome of at least 10 nucleotides is located, and is located in a SARS-CoV-2—a nucleotide sequence at least 10 nucleotides of the variable region CoV-2 genome or a nucleotide sequence located at least 10 nucleotides of the protein-encoding structural gene of the genome of SARS-CoV-2 in, and located in a SARS-CoV-2 nucleotide sequence at least 10 nucleotides of the non-structural protein-coding genes in the genome—CoV-2; b) with a sample containing or suspected of containing the nucleotide sequence of the SARS-CoV-2 contacting the kit under conditions suitable for nucleic acid hybridization; and c) evaluating the SARS-CoV-2 nucleotide sequence (if present in the sample), and i) a nucleotide sequence located respectively SARS-CoV-2 genome complementary to conserved regions of the oligonucleotide nucleotide probe, a nucleotide sequence and a variable region of the SARS-CoV-2 genome of said oligonucleotide probe is complementary to, or ii) a structural protein coding gene located in the genome of SARS-CoV-2 in a nucleotide sequence complementary to Said oligonucleotide hybridization complex formed between the nucleotide probe, and the oligonucleotide probe a nucleotide sequence of the gene encoding the non-structural proteins located in SARS-CoV-2 genome complementary to determine the sample SARS-CoV-2 in the presence or absence or quantity, so long as the detection of one or both of the hybridization complex indicates the presence on the SARS-CoV-2 in the sample.

In another particular embodiment, the method of the present invention comprises: a) providing a kit that includes an oligonucleotide probe complementary to a nucleotide sequence located on the genome of SARS-CoV-2 conserved region, a oligonucleotide probe nucleotide sequences of the variable region of the SARS-CoV-2 genome complementary to, at least oligonucleotide probes in three of the following: an immobilized control probe, the probe is labeled, when containing or suspected of containing the kit sample of SARS-CoV-2 in contact with, the probe does not participate in any hybridization reaction, a positive control probe, which any sequence is not complementary to SARS-CoV-2, but non-SARS-CoV-2 in the sample containing the sequence complementary to a negative control probe, comprising any nucleotide sequence which is not complementary to the sample, and a blank point; b) containing or suspected of containing SARS-CoV-2 nucleoside contacting the sample kit acid sequence under conditions suitable for nucleic acid hybridization; and c) evaluation: i) (if present in the sample), respectively, and conservative and SARS-CoV-2 genome in a nucleotide sequence of the SARS-CoV-2 area nucleoside Hybrid complexes formed between the sequence complementary to the oligonucleotide probes and oligonucleotide probes of the variable region of the nucleotide sequence of the genome of SARS-CoV-2 in complementary; ii) comprises a control probe in the immobilized the marker, or positive control probe directed and/or negative control probe hybridization complex; and iii) a signal point on the blank, to determine the presence in the sample or not, or a SARS-CoV-2 number.

Preferably, the present invention includes a kit located two of SARS-CoV-2 replicase 1A or 1B two different gene nucleotide sequences complementary to the oligonucleotide probes, located a gene of SARS-CoV-2 N nucleotide sequence complementary to an oligonucleotide probe complementary to a nucleotide sequence located in the SARS-CoV-2 S gene oligonucleotide probes, control probes immobilized one, a positive control probe needle and a negative control probe, when there exists a case may be determined SARS-CoV-2: a) at least one nucleotide located using two different nucleotide sequences of SARS-CoV-2 replicase 1A or 1B gene of acid probes, and the nucleotide sequence of SARS-CoV-2 is located in the N gene oligonucleotide probes complementary to the nucleotide sequence and positioned in the SARS-CoV-2 S gene complementary oligonucleotide probes, A positive hybridization signal is detected; b) detecting the immobilized control probes positive signals; c) the positive control probes to detect positive hybridization signals; d) using a negative control probe positive hybridization signals cannot be detected; and e) at point blank, does not detect positive hybridization signals.

Comprising a target sequence can be evaluated for possible mutations, SARS-CoV-2 in the variable region of SARS-CoV-2. For example, the use of two different nucleotide sequences located SARS-CoV-2 replicase 1A or 1B, at least one nucleotide sequence is located or with the SARS-CoV-2 N gene oligonucleotide probes complementary to needle, positive hybridization signals detected; the use of oligonucleotide probes complementary to the detection of SARS-CoV-2 located in the nucleotide sequence of the S gene not positive hybridization signals, suggesting that the mutation of SARS-CoV-2.

The method of the present invention may be any suitable prognostic and diagnostic purposes. In one embodiment, the method of the present invention are used to confirm the detection of patients infected with SARS-CoV-2, wherein the COVID-19-like symptoms such as fever or high fever, cough without sputum, myalgia, patient groups having from COVID-19-like symptoms of difficulty in breathing, high lactate dehydrogenase, hypocalcemia and lymphopenia (Guan W, et al. Clinical characteristics of coronavirus disease 2019 in China. N Engl J Med 2020; 382:1708-1720. DOI: 10.10056/NEJMoa2002032.). Kit of the present invention, methods and kits may further comprise detecting a high lactate dehydrogenase, hypocalcemia and lymphopenia and the like.

In another embodiment, the use of such a kit, i.e., the kit further comprises a nucleotide sequence of oligonucleotide probes that do not involve the SARS-CoV-2 genome complementary, the method is used for already infected patients, for example, influenza A virus and/or influenza B virus. The patients also infected SARS-CoV-2 later.

Supply Sample 3 has showed that IFA infects lymphocytes and could destroy immune systems in human. Therefore, anti-influenza medication can save life of patients. It has been suggested that all patients should be tested SARS-CoV-2, influenza A virus, influenza B virus every week in hospitals to early define the co-infection in hospitals using RT-PCR (SARS-CoV-2, influenza A virus, and influenza B vino) or ELISA (IgM antibodies to SARS-CoV-2, influenza A virus, and influenza B virus).

Assays for a wide variety of single point substitutions, insertions, or deletions (Table 1). A widely used strategy for detecting DNA sequence variants is allele-specific PCR in which one or both primers are designed to anneal at sites of sequence variation. Ideally, a primer whose sequence matches a specific variant should selectively amplify only that variant; however, in practice, significant mismatched amplification typically occurs. It is common practice to anchor the 3′ end of the allele-specific primer at the mutant base in order to selectively amplify the mutant template. This strategy reduces but does not eliminate amplification of the wild-type allele. The amount of this non-specific amplification has been found to vary widely depending on the particular base mismatch between the allele-specific primer and the wild-type sequence. The variability of nonspecific amplification typically requires a process of trial and error when designing highly selective mutation assays. The assay method reported here utilizes a combination of allele-specific PCR primers, a blocker oligonucleotide to suppress amplification of the wild type allele, and a set of reagent design rules that consistently produce highly selective assays for a wide variety of single point substitutions, insertions, or deletions. We refer to the modified assay by the acronym ASB-PCR (Allele-Specific Blocker PCR). Features of the method include the ability to detect mutations in either DNA or RNA with a high level of sensitivity and selectivity. No proprietary reagents are required so the method can be performed in any laboratory with real-time PCR capability. Moreover, the assay is compatible with the process stream for real-time RT-PCR, enabling mutation analysis to be carried out alongside gene expression analysis of the same clinical specimen.

Sequences in Table 1 represented by SEQ ID NO: 1 with the mutation C241A, 5′UTR referred to as COIN-CoV2-241; SEQ ID NO: 2 with mutation C3037T, ORF1ab referred to as COIN-CoV2-3037; SEQ ID NO: 3 with mutation C14408A, ORF1ab referred to as COIN-CoV2-14408; SEQ ID NO: 4 with mutation T28144C, ORF8 referred to as COIN-CoV2-28144; SEQ ID NO: 5 with mutation A23403C, Protein S referred to as COIN-CoV2-23403; SEQ ID NO: 6 with mutation G28881 A, Protein N referred to as COIN-CoV2-28881; SEQ ID NO: 7 with mutation G28882T, Protein N referred to as COIN-CoV2-28882; SEQ ID NO: 8 with mutation G28883A, Protein N referred to as COIN-CoV2-28883; SEQ ID NO: 9 with mutation C17747T, Protein NSP13 referred to as COIN-CoV2-17747; SEQ ID NO: 10 with mutation C238A, ORF referred to as COIN-WA-238; and SEQ ID NO: 11 and/or SEQ ID NO: 12 collectively referred to as COIN-IFB-002 with an influenza B viruses (“B/Victoria/2/87-like”) mutation at hemagglutinin (HA) protein at D129G or R498K could be used for identifying single nucleotidepoly morphism (SNPs). The SNP mutations, including nucleotide changes and the corresponding positions in a genome, are called an SNP profile. The SNP profiles of SARS-CoV-2, IFA or IVB isolates are retrieved and parsed from the aligned genomes according to the reference genome SARS-CoV-2, IFA or IVB. The SNP profile of the complete genome of a virus can be considered as the genotype of the virus. An SNP profile could be identified by Mass Spectrometry described as following:

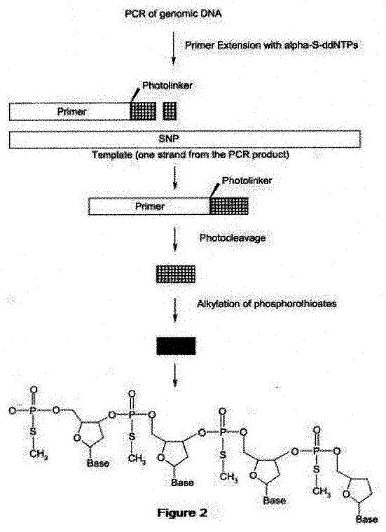

The principle of the procedure starting with a PCR followed by allele-specific primer extension reaction with a primer containing a photolinker and phosphorothioates at the 3′-end. The unmodified part of the primer is cut off by exposure to UV light and the modified part is then allylated. The resulting product contains a DNA backbone with one negative charge deriving from the 5′-end. Thus, the product is negatively charge-tagged. Products are diluted and transferred onto a MALDI target for analysis (FIG. 2).

Synthetic RNA templates for selected mutations were prepared using a method for generating long templates from shorter oligonucleotides: Two synthetic oligonucleotides were designed to be partially complementary at their 3′ ends and have a combined length that encompassed the mutation assay amplicon. The oligonucleotides were denatured at 95° C. for three minutes and then cooled rapidly on ice. The products were extended in a Klenow reaction containing 25 pmol annealed oligonucleotides, 5 Units Klenow Fragment (New England Biolabs, Ipswich, Mass.), 1 mM dNTPs and 16 NEB2 Buffer (New England Biolabs). A MegaShortscript™ IVT reaction (Applied Biosystems/Ambion) was performed in 20 mL according to manufacturer's instructions with one mL of a 1:5 dilution of Klenow reaction. The IVT reaction was run at 37° C. overnight followed by treatment with 1 mL DNAseI at 37° C. for 15 minutes. Reactions were purified with an RNEasyH Kit (QIAGEN) and IVT yield was determined by A260 and confirmed by limiting dilution TaqManH assay. A dilute synthetic DNA oligonucleotide carrying the mutation G215C was geted from Eurogentec North America (San Diego, Calif.). Concentration was verified by limiting dilution TaqManH assay. Oligonucleotide primers and probes were geted from Integrated DNA Technologies (Coralville, Iowa).

TaqMan RT-PCR Assays. Reverse transcription was performed using an OmniScript RT Kit (QIAGEN) according to the manufacturer's instructions in a 10 mL volume with 50 nM of each reverse primer. TaqMan PCR was performed with an RT volume of up to 1.25 mL in a 5 mL assay with 16TaqMan Universal PCR Master Mix (no UNG)™ (Applied Biosystems, Foster City, Calif.), 900 nM primers, 200 nM probe and 3600 nM blocker. One ng of RNA or 0.4 ng of DNA extracted from FPET were analyzed in each PCR reaction, unless noted otherwise. Standard TaqMan thermocycling conditions were used: 10 min. at 95 uC, 40 cycles of 20 sec. at 95 uC, 45 sec. at 60 uC. All PCR assays were run in triplicate or at higher replication when deemed necessary. A list of the oligonucleotides used for all of the PCR mutation assays is provided in Table 1.

Assays that use the forward primer as the discriminating primer are appended with “0.1” and assays that use the reverse primer in this fashion are appended with “0.2”. PrimerExpress™ (Applied Biosystems, Foster City, Calif.) was used for assay design and estimation of oligonucleotide melting temperature.

Sequence Analysis. Bi-directional sequencing (one pass per direction) was performed by SeqWright (Houston, Tex.) on 53 ProteoGenex FPE tissue gDNA samples using dye-terminator chemistry (ABI BigDyeH v3.1) on an ABI 3730xl DNA sequencer. Traces were aligned to a reference sequence and identical results were required in both forward and reverse sequencing strands to make minor base calls. In the case where only one sequence trace was available minor alleles were called if the secondary peak was greater than 20% of the primary peak In all cases calling a minor allele as present was weighed against the amount of noise in the immediate vicinity of the peak of interest.

To accomplish allele-specific PCR, two criteria were introduced into our assay designs. First the mutant-specific primer (FIG. 3) was shortened at its 5′-end to reduce its Tm to approximately 10° C. below the anneal/extend temperature of the assay. Second, a blocking oligonucleotide, complementary to the wild type sequence but phosphorylated at the 3′-end to prevent extension, was added to further suppress nonspecific amplification of the wild type allele by the mutant-specific primer. The blocking reagent was designed to have the variant base position approximately in the middle of the oligonucleotide and to partially overlap the sequence of the mutantspecific primer. Two additional reagents, a second PCRprimer and a Taqman probe, are needed for real-time PCR of either the wild type or variant template. With reference to this, Table 2 provides exemplary human SARS-CoV-2 primer, where SYBR Green SARS-COV2-FOR Primer 1 or SEQ ID NO: 13 is the first forward primer; SYBR Green SARS-COV2-REV Primer 1 or SEQ ID NO: 14 is the first reverse primer; SYBR Green SARS-COV2-FOR Primer 2 or SEQ ID NO: 15 is the second forward primer; SYBR Green SARS-COV2-REV Primer 2 or SEQ ID NO: 16 is the second reverse primer; Dual Labeled probe 1 SARS-COV2-FOR Primer 1 or SEQ ID NO: 17 is the first dual labeled forward primer; Dual Labeled probe 1 SARS-COV2-REV Primer 1 or SEQ ID NO: 18 is the first dual labeled reverse primer; Dual labeled probe 1 or SEQ ID NO: 19 is the first dual labeled probe; Dual Labeled probe 2 SARS-COV2-FOR Primer 2 or SEQ ID NO: 20 is the second dual labeled forward primer; Dual Labeled probe 2 SARS-COV2-REV Primer 2 or SEQ ID NO: 21 is the second dual labeled reverse primer; Dual labeled probe 2 or SEQ ID NO: 22 is the second dual labeled probe; Dual Labeled probe 3 SARS-COV2-FOR Primer 3 or SEQ ID NO: 23 is the third dual labeled forward primer; Dual Labeled probe 3 SARS-COV2-REV Primer 3 or SEQ ID NO: 24 is the third dual labeled reverse primer; Dual labeled probe 3 or SEQ ID NO: 25 is the third dual labeled probe; Dual Labeled probe 4 SARS-COV2-FOR Primer 4 or SEQ ID NO: 26 is the fourth dual labeled forward primer; Dual Labeled probe 4 SARS-COV2-REV Primer 4 or SEQ ID NO: 27 is the fourth dual labeled reverse primer; Dual labeled probe 4 or SEQ ID NO: 28 is the fourth dual labeled probe. Table 3 provides exemplary influenza A virus primer, where SEQ ID NO: 29 is Influenza A virus-FOR Primer 1 or first forward primer; SEQ ID NO: 30 is Influenza A virus-REV Primer 1 or first reverse primer; SEQ ID NO: 31 is Influenza A virus-FOR Primer 2 or second forward primer; SEQ ID NO: 32 is Influenza A virus-REV Primer 2 or second reverse primer; SEQ ID NO: 33 is Influenza A virus-FOR Primer 3 or third forward primer; SEQ ID NO: 34 is Influenza A virus-REV Primer 3 or third reverse primer; SEQ ID NO: 35 is Influenza A virus-FOR Primer 4 or fourth forward primer; and SEQ ID NO: 36 is Influenza A virus-REV Primer 4 or fourth reverse primer. And, Table 4 provides exemplary influenza B virus primer, where SEQ ID NO: 37 is Influenza B virus-FOR Primer 1 or first forward primer; SEQ ID NO: 38 is Influenza B virus-REV Primer 1 or first reverse primer; SEQ ID NO: 39 is Influenza B virus-FOR Primer 2 or second forward primer; SEQ ID NO: 40 is Influenza B virus-REV Primer 2 or second reverse primer.

The detection of the SARS-CoV-2 patient from a blood sample show detecting SARS-CoV-2, IFA or IFB from a patient blood sample. Lymphocytes were isolated. RNA was extracted using the Viral NA/gDNA Kit on the chemagic 360 system (PerkinElmer) using the automated chemagic 360 instrument (PerkinElmer) or manually using the Qiagen Viral RNA Mini Kit (Qiagen).

To detect the SARS-CoV-2 virus by PCR, the Towpaths COVID-19 CE-ID RT-PCR Kit (Life Technologies) was used according to the manufacturer's instructions. The assays target genomic regions (ORF1ab, S protein and N protein) of the SARS-CoV-2 genome. RT-PCR was performed on a QuantStudio 7 Flex Real-Time PCR instrument (Life Technologies). Ct values were analyzed using auto-analysis settings with the threshold lines falling within the exponential phase of the fluorescence curves and above any background signal.

Complementary DNA (cDNA) synthesis was performed on the RNA using random primers followed by gene-specific multiplex PCR using the ARTIC protocol. Briefly, extracted RNA was converted to cDNA using the Superscript IV First Strand synthesis system (Life Technologies) and random hexamer primers. SARS-CoV-2 whole-genome amplification by multiplex PCR was carried out using primers designed on Primal Scheme (http://primal.zibraproject.org/) to generate 400-bp amplicons with an overlap of 70 bp that covers the 30-kb SARS-CoV-2 genome. PCR products were cleaned up using AMPure XP purification beads (Beckman Coulter) and quantified using the Qubit dsDNA High Sensitivity assay on the Qubit 4.0 instrument (Life Technologies).

The Illumina Nextera Flex DNA Library Prep Kit was used according to the manufacturer's protocol to prepare uniquely indexed paired-end libraries of genomic DNA. Sequencing libraries were normalized to 4 nM, pooled and denatured with 0.2 N sodium acetate. A 12 pM sample library was spiked with 1% PhiX (PhiX Control v3 adapter-ligated library used as a control). Libraries were loaded onto a 500-cycle v2 MiSeq Reagent Kit and run on the Illumina MiSeq instrument (Illumina).

Raw reads coming from Illumina sequencing were assembled using Genome Detective 1.126 (https://www.genomedetective.com/) and the Coronavirus Typing Tool31-32. The initial assembly geted from Genome Detective was polished by aligning mapped reads to the references and filtering out low-quality mutations using the bcftools 1.7-2 mpileup method. All mutations were confirmed visually with BAM files using Geneious software (Biomatters).

In still another embodiment, the use of such a kit that comprises a nucleotide sequence of oligonucleotide probes are highly expressed SARS-CoV-2 genome complementary to, the method is used for the early diagnosis of patients with COVID-19, for example, it has been infected with SARS-CoV-2 patients about less than a day to about three days of SARS-CoV-2.

In still another embodiment, the present invention is a method for monitoring the treatment of COVID-19, for example, treatment with a variety of RNA virus replication inhibition ribavirin or interferon such reagents. The method of the present invention can also be used to evaluate potential anti-SARS-CoV-2 agents in drug screening experiments.

The method of the present invention may be used to determine whether a subject is infected with SARS-CoV-2 and/or cause COVID-19-like symptoms of SARS-CoV-2 infection of non-biological. SARS-like symptoms leading to non-SARS-CoV-2 infection organisms including but not limited to IFA or IFB.

Any suitable non-SARS-CoV-2 or SARS-CoV-2 infection biological nucleotide sequences can be detected. For example, the detected biological SARS-CoV-2 or nucleotide sequence may be a SARS-CoV-2 infection SARS-CoV-2 RNA or SARS-CoV-2 infection biological genomic sequences, or infection from the genome of a RNA extracted SARS-CoV-2 or SARS-CoV-2 DNA sequence of the amplified sequences.

SARS-CoV-2 infection in an RNA or biological SARS-CoV-2 genome sequence can be prepared by any suitable method. E.g., SARS-CoV-2 RNA or SARS-CoV-2 infection genome of an organism using QIAamp Viral RNA sequences can kit, Chomczynski-Sacchi technique or TRIzol (De Paula et al, J. Virol. Methods, 98 (2): 119-25; 2001) infected with the SARS-CoV-2 or non-SARS-CoV-2 infection in cells or other biological materials extraction. Preferably, SARS-CoV-2 RNA or non-SARS-CoV-2 infection the genome sequence using QIAamp Viral RNA kit or SARS-CoV-2 infection of non-SARS-CoV-2 infection in cells or other biological material from the extract. SARS-CoV-2 infection in an RNA or biological SARS-CoV-2 genome sequence can be extracted from any suitable source. E.g., SARS-CoV-2 RNA or SARS-CoV-2 infection sequence of the genome can be extracted from sputum or saliva sample. Embodiment, SARS-CoV-2 RNA or SARS-CoV-2 infection biological genomic sequences can be extracted from a blood sample lymphocytes in another embodiment.

SARS-CoV-2 RNA or biological SARS-CoV-2 infection genomic sequences can be amplified by any suitable method, such as PCR. Preferably, the label is incorporated during PCR amplification of DNA sequences. Any suitable PCR can be used, such as conventional PCR, multiplex PCR, nested PCR or RT-PCR. In one embodiment, PCR can include a two-step nested PCR, the first step is a RT-PCR, the second step is a conventional PCR. In another embodiment, the PCR may comprise a step multiplex RT-PCR, which uses multiple 5′ and 3′ specific primers, wherein each primer comprises a specific thereto a sequence complementary to the amplified target specific sequence and a common sequence, and a 5′ and 3′ universal primers, wherein the 5′ universal primer with a 5′ specific primer consensus sequence is complementary to and a common sequence ‘universal primer 3’ specific primer complementary to, wherein PCR, the 5′ and 3′ universal primer concentrations are equal to or higher than the concentration of 5′ and 3′ specific primers. Preferably, the 3′ universal primer and/or a 5′ universal primer is labeled, for example a fluorescent label. In still another embodiment, a multi-step PCR including nested PCR or RT-PCR. In still another embodiment, at least the PCR using the following primers in a primer pair to be, the following primer pairs for SARS-CoV-2.

In still another embodiment, the PCR is using the following primers in the at least one primer pair for pair, wherein the following primers for the non-infected organisms SARS-CoV-2 cause a COVID-19-like symptoms shown in Table 1.

In a particular embodiment, the present invention relates to a kit for amplification of SARS-CoV-2 or non-SARS-CoV-2 infection in the biological nucleotide sequence, the kit comprising: a) a primer as described above; and b) may be used to amplify a probe SARS-CoV-2 or non-SARS-CoV-2 infection biological nucleotide sequence of a nucleic acid polymerase. Preferably, the nucleic acid polymerase is a reverse transcriptase.

In still another aspect, the present invention relates to oligonucleotide probes, hybridizing to nucleotide sequences for biological SARS-CoV-2 or non-SARS-CoV-2 infection, the oligonucleotide probe comprises a nucleotide having the following features acid sequences: a) under high stringency conditions biological target nucleotide sequence or its complementary strand with the SARS-CoV-2, IFA or IFB; or b) comprises the nucleotide sequence of SARS-CoV-2, IFA or IFB infection biological target nucleotide sequence or its complementary strand having at least 90% identity.

In a particular embodiment, the present invention relates to a kit for SARS-CoV-2 and/or hybridization analysis of SARS-CoV-2 infection biological nucleotide sequence, the kit comprising: a) a as described above probe; and b) a method for evaluation of SARS-CoV-2 and/or SARS-CoV-2 infection of hybridization complexes between biological nucleotide sequence and the probe is formed.

Oligonucleotide primers and probes may be produced by any suitable method. For example, probes can be chemically synthesized (see generally, Ausubel (ed.) Current Protocols in Molecular Biology, Synthesis and purification of oligonucleotides, John Wiley & amp; Sons, Inc (2000).), isolated from natural sources and produced by recombinant methods, or combinations thereof. Synthetic oligonucleotides may be used by Matteucci et al., J. Am. Chem. Soc, Triester method of preparing 3185-3191(1981). Optionally, the automated synthesis is preferred, for example, in the use cyanoethyl phosphoramidite chemistry Applied Biosynthesis DNA synthesizer. Preferably, probes and primers are chemically synthesized.

Suitable bases of the present invention, oligonucleotide probes and primers may be selected from naturally occurring nucleotide bases of white, such as adenine, cytosine, guanine, uracil and thymine. It may also be selected from non-naturally occurring or “synthetic” nucleotide bases such as 8-oxo-guanine, 6-thioguanine, 4-acetyl cytidine, 5-(carboxy-hydroxyethyl) uridine, 2′-O-methyl cytidine, 5-carboxymethylamino-methyl-2-thioridine, 5-carboxy-methylamino-methyluridine, dihydrouridine, 2′-O-methylpseudouridine, β-D-galactosyl queosine, 2′-O-methyl guanosine, inosine, N6-isopentenyl adenosine, 1-methyl adenosine, 1-methyl pseudouridine, 1-methyl guanosine, 1-methyl-inosine, 2,2-dimethyl-guanosine, 2-methyl adenosine, guanosine 2-methyl, 3-methyl cytidine, 5-methylcytidine, N6-methyladenosine, 7-methylguanosine, 5-methyl-aminomethyl uridine, 5-methoxy amino methyl-2-thiouridine, β-D-mannosyl queosine, 5-A butoxycarbonyl-methyluridine, 5-methoxy-uridine, 2-methylthio-N6-isopentenyl adenosine, N-((9-β-D-ribofuranosyl-2-methyl thio-purin-6-yl) carbamoyl) threonine, N-((9-β-D-ribofuranosyl-purin-6-yl) N-methylcarbamoyl) threonine, uridine-5-oxo-acetic acid methyl ester, uridine-5-oxyacetic acid, wybutoxosine, pseudouracil, pseudouridine, queosine, 2-sulfur Substituting cytidine, 5-methyl-2-thiouridine, 2-thiouridine, 2-thiouridine, 5-methyluridine, N-((9-β-D-ribofuranosyl purin-6-yl) carbamoyl) threonine, 2′-O-methyl-5-methyluridine, 2′-O-methyluridine, wybutosine, and 3-(3-amino-3-carboxypropyl) uridine.

Likewise, the oligonucleotide may be used as a chemical analogue (e.g., wherein the phosphodiester bond has been modified oligonucleotides such modifications as methyl phosphate, phosphotriester, phosphorothioate, dithiophosphoric phosphate or phosphoramidate (phosphoramidate)). It can be “3 terminal cap” strategy to achieve protection from degradation by the nuclease resistant policy key enzyme of the oligonucleotide 3′ by using a substituted phosphodiester bond (Shaw et al., On the end, Nucleic Acids Res., 19: 747 (1991)). Phosphoramidates, phosphorothioates and methyl phosphonates key, in this manner function adequately. More extensive modification of the phosphodiester backbone has been shown to impart stability, and may allow enhanced affinity oligonucleotides and increased cell infiltration (Milligan, et al., J. Med. Chem, 36: 1923 (1993)). It has been used in many different chemical strategies to complete a substituted phosphodiester backbone with novel linkages. Backbone analogues include phosphorothioate, phosphorodithioate, methylphosphonate, phosphoramidate, boranophosphate, phosphate triester, formacetal, 3′-thioformacetal, 5′-thioformacetal, 5′-thioether, carbonate ester, 5′-N— carbamate, sulfate, sulfonate, sulfamate, sulfonamide, sulfone, sulfite, sulfoxide, sulfide, hydroxylamine, methylene (methylimino) (the MMI) or a methyleneoxy (methylimino) (MOMI) key. Methylphosphonate and phosphorothioate modified oligonucleotides are particularly preferred, since they can be synthesized by automated oligonucleotide geted nucleotide. Oligonucleotides may be a “peptide nucleic acid”, as described by (Milligan, et al., J. Med. Chem, 36: 1923 (1993)) described herein. The only requirement is that the oligonucleotide probe should have such a sequence, i.e., at least a portion of the sequence capable of binding to the target sequence with a portion of SARS-CoV-2 sequences.

Hybridization probe or amplification primer may be any suitable length. No lower or upper limit to the length of the probe or primer, as long as the probe is SARS-CoV-2, IFA or IFB infection biological target nucleic acid, and can be used as probes or primers to function effectively (e.g. remove or facilitate amplification). Probes and primers of the present invention may be as short 50, 40, 30, 20, 15 or 10 nucleotides or less. Similarly, the probe or primer can be as long 20, 40, 50, 60, 75, 100 or 200 nucleotides or longer, for example, as long as the full-length SARS-CoV-2, IFA or IFB infection in a biological target sequence. Generally, the probe having a complementary target nucleic acid strand in a chain of any of at least 14 nucleotides, preferably at least 18 nucleotides, more preferably at least 20 to 30 nucleotides, and does not include any fat two folder structures. In a particular embodiment, the length of the probe is at least 30 nucleotides or at least 50 nucleotides. If having complete complementarity, i.e., if the chain has the same sequence as a probe sequence, the duplex under even stringent conditions will be relatively stable, and the probes may be short, i.e., in the range of about 10 to 30 base pairs. If the expected some degree of mismatch in the probe, i.e., if the probe is expected to hybridize to the variable region, or a group of sequences such as all species hybridization specificity of the species, the probe may be longer (i.e. 15-40 bases) to balance the effects of mismatches. The probe does not need to span the entire SARS-CoV-2, IFA or IFB infection biological target gene. May be specifically detect SARS-CoV-2 or any subset of SARS-CoV-2 infection of a target region or allele biological target substance (allele) using potential. Thus, the target region of nucleic acid probes may be as few as 8 nucleotides hybridize. Further, the probe fragments may be used, as long as these fragments have sufficient characteristics, distinguish the classified SARS-CoV-2, IFA or IFB infection biological target gene region.

You should be able to probe or primer length of at least 8 nucleotides of SARS-CoV-2, IFA or IFB infection biological target nucleotide sequence hybridizes under low stringency. Preferably, the probe or primer with a biological or non-infectious SARS-CoV-2 target nucleotide sequence hybridizes under medium stringency or high stringency.

In still another aspect, the present invention relates to an immobilized in an array on a support oligonucleotide probes, or for determining the type of SARS-CoV-2 infection of SARS-CoV-2 target gene organisms, the array comprising a support, suitable for use in nucleic acid hybridization, the support may be fixed on a plurality of oligonucleotide probes, said probe comprising at least a nucleotide sequence having the following features: a) at high stringency SARS-CoV-2 target or non-biological SARS-CoV-2 infection or a nucleotide sequence complementary strand; or b) comprises a target nucleotide sequence SARS-CoV-2, IFA or IFB infection biological nucleotide sequence or its complementary strand having at least 90% identity.

The probe may include a variety of DNA, RNA, PNA or derivatives thereof. At least some of the probes may comprise a table or a nucleotide sequence or its complementary strand. Preferably, the probe array includes all nucleotide sequences or its complementary strand. At least one, some or all of probes may be labeled. Exemplary labels include chemical, enzymatic, immunochemical, radioactive, fluorescent, luminescent and a FRET label. Any suitable support may be used in the kit of the present invention, for example, silicon, plastic, glass, ceramics, rubber, and the polymer surface.

Immobilization methods of the invention, probes and probe arrays E. probe detection format may be used in solution. Preferably, in a kit format, such as use a probe immobilized on a solid support.

The probe may be immobilized on any suitable surface, preferably on a solid support, such as silicon, plastic, glass, ceramics, rubber, or polymer surface. The probe may also be fixed three-dimensional porous substrate in a gel, e.g. Packard HydroGel kit (Broude et al., Nucleic Acids Res, 29 (19):E92 [2001]).

For array-based detection, the probe is preferably immobilized on a solid support, such as a “Bio-kit.” The solid support may be biological, nonbiological, organic, inorganic or any combination of these, a particle, strand, precipitate, gel, sheet, tubing, spheres, containers, capillaries, pads, slices, films, there are plates, slides and other forms.

Library comprises a probe microarray Bio-kit can be prepared by methods well known in the plurality of, for example, these methods include a light control method are also suitable for the preparation of a library of hairpin probes in the microarray, this patent describes in detail the different libraries prepared as double-stranded probes using a microarray of VLSIPS™.

The method may also be used to spot the preparation of a microarray Bio-kit on which a plurality of probes may be fixed. In this case, the reaction was purified by direct deposition of a relatively small amount in the selected area of the support is transmitted. In some steps, of course, the entire surface of the support may be sprayed with a specific solution, or in addition with a specific coating solution. In a particular form, the dispenser (a dispenser) moves between the regions in a particular format, with each deposited only necessary to stop as many probes or other reagents. Typical dispensers include a micropipette, nano pipette, an ink jet cartridge and needle to a solution containing a probe or other liquid is transferred to the support, optionally, comprise a robotic system to control the delivery device the location of the support. In other formats, the dispenser includes a series of tubes, a series of pipes and transfer equipment, so that more of the reactants can be simultaneously transferred to the reaction zone. In some cases, the liquid flow path, and “spot” on a combination of a predetermined area of the support may also be used to prepare a microarray having the probe immobilized Bio-kit.

The solid support for fixation of the probe is preferably flat, but may have alternative surface configurations. For example, the solid support may contain raised or depressed regions, probe synthesis occurs in these areas, or probe bound to these regions. In some embodiments, the solid support may be selected to provide the appropriate light adsorption characteristics. For example, the support may be a polymerized Langmuir Blodgett film, functionalized glass, or glass, Si, Ge, GaP, SiO2, SiN4, modified silicon, or more gels or polymers such as (poly) tetrafluoroethylene, (poly) vinylidene fluoride ((poly) vinylidendifluoride), polystyrene, polycarbonate any one, or a combination thereof. Other suitable solid support materials to those of ordinary skill in the art will be apparent.

Surface of the solid support may contain reactive groups, such reactive groups include carboxyl, amino, hydroxyl, thiol group or the like, suitable reactive group conjugated with oligonucleotide or nucleic acid-related. Preferably, the surface is optically transparent and will have surface Si—OH functionalities, such as those found on the functionality of the silicon surface.

Probes may be other forces, such as by ionic, covalent, or by chemical or physical means well known in the art is attached to the support. Any method of nucleic acids and oligonucleotides can be immobilized by techniques well known in the art to achieve (e.g., participants, Dattagupta et al., Analytical Biochemistry, 177: 85-89 (1989); Saiki et al., Proc. Natl. Acad. Sci. USA, 86: 6230-6234 (1989); and Gravitt et al., J. Clin. Micro, 36: 3020-3027 (1998)).

Probes may be attached by way of a spacer molecule to a support, which may be useful in hybridization experiments. Spacer molecule typically comprises a length of between 6-50 atoms, including the attachment portion to a support surface. Supports attached to the carbon-carbon bond may be achieved by, for example, having a (poly) vinyl chloride support surface trifluoromethyl, or preferably achieved by a siloxane bond (e.g., glass or silica as the solid support). Siloxane bonds can be formed by the reaction between the support and the spacer trichlorosilane group or trialkoxy silyl groups. Hydroxyalkyl silanes and aminoalkyl silanes, bis (2-hydroxyethyl)-amino propyl triethoxysilane, 2-hydroxyethyl-aminopropyl triethoxysilane, aminopropyl triethoxysilane or hydroxypropyl triethoxysilane useful surface attachment group.

Also includes spacer groups attached to the attachment portion of the extending portion of the surface of the probe or longer chain moiety. For example, an amino group, a hydroxyl group, a thiol group and a carboxyl group adapted to the extended portion of the spacer is attached to the upper surface of the attachment portion. The extended portion of the spacer may be any of a variety of molecules, these molecules of any subsequent polymer synthesis conditions there is no activity. The longer chain moiety is typically ethynyl aryl group containing 2-14 monomer units of ethylene glycol oligomers, diamines, diacids, amino acids, peptides, or combinations thereof.

In some embodiments, the extended portion of the spacer is a polynucleotide, or the entire spacer can be a polynucleotide. The extended portion of the spacer may be made of polyethylene glycol, a polynucleotide, alkylene, polyol, polyester, polyamine, acid diesters, and combinations thereof. Further, for use in the synthesis of the probe, the spacer may be spacer (corresponding to a solid support) of the distal end or tip having a protected functional group is attached to (e.g. hydroxy, amino or carboxylic acid) on group. After deprotection and coupling, the distal end may be covalently bonded to the oligomer or probe.

The method of the present invention may be used to analyze each single sample having a single probe. Preferably, the process is performed in a high throughput format. For example, we can use a single probe analysis of multiple samples simultaneously with a plurality of probes or simultaneous analysis of single sample. More preferably, multiple samples can be analyzed simultaneously using a plurality of probes.

Any suitable technique for the conditions of the hybridization conditions is known in the art. Of ordinary skill in the art will be apparent that hybridization conditions may be changed to increase or decrease the degree of hybridization, hybridization specificity level, background level of nonspecific binding (i.e., by changing the salt concentration or temperature of the hybridization or wash). Hybridization between the probe and the target nucleotide sequence can be conducted at any suitable stringency, including high, medium or low stringency. Typically, hybridization performed under conditions of high stringency.

Between the probe and the target nucleic acid can be homologous, for example in molecular signaling (Tyagi S et al., Nature Biotechnology, 14: 303-308 (1996)), and the hybridization protection assay (GEN-Probe, Inc) typical conditions is used, or typical conditions (using nitrocellulose-based hybridization of different types of heterologous and those conditions used in the hybridization beads).

By a target polynucleotide sequence under high to low stringency hybridization and wash conditions detected by hybridization to an oligonucleotide probe sequence of the oligonucleotide probe to the target sequence to form a stable hybridization complex. An advantage of detection by hybridization is that, depending on the probe used, it is possible to further specificity. If the probe to the target can be expected to be fully complementary sequence (i.e., about 99% or more), it is high stringency conditions. If some mismatching is expected, for example, if variant strains of such a desired result, i.e., the probe is not perfectly complementary, the stringency of the hybridization can be reduced. However, the conditions are selected so as to minimize or eliminate non-specific hybridization.

Those conditions and the conditions affect hybridization against non-specific hybridization in the present selection is known in the art (Molecular Cloning A Laboratory Manual, second edition, J. Sambrook, Cold Spring Harbor Laboratory Press, 1989). Generally, lower salt concentration and higher temperature increase the stringency of hybridization. For example, in general, stringent hybridization conditions include containing about 0.1×SSC, 0.1% SDS solution, and incubated at about 65 deg.] C incubation/wash temperature. In stringency conditions in a solution containing about 1-2×SSC, 0.1% SDS solution, and incubated at about 50° C.-65° C. incubation/wash temperature. Low stringency conditions are 2×SSC and about 30° C.-50° C.

An optional method first hybridization and wash is low stringency hybridization (5×SSPE, 0.5% SDS), then in the presence of 3M tetramethyl—washed under high stringency chloride (of TMAC) case. TMAC is the compensation effect of AT and GC base pairs relative binding, so that the efficiency of hybridization at a given temperature corresponds more closely to the length of the polynucleotide. Use TMAC, washing temperature may be varied to achieve the desired level of stringency (Wood et al., Proc. Natl. Acad. Sci. USA, 82: 1585-1588 (1985)).

Hybridization solution is containing 25% formamide, 5×SSC, 5XD enhardt's solution, single-stranded DNA 100 μg/ml of 5% dextran sulfate, or other agents known to be useful for probe hybridization.

Detecting hybrids in any method of detecting hybridization between the probe body and the SARS-CoV-2 or IFA or IFB target nucleic acid sequence can be known in the art, for example a labeled probe, labeled second probe, labeled target nucleic acid, or some combinations of these methods are suitable for the purposes of the present invention. Further, in the absence of a detectable marker, the hybrid can be detected by mass spectrometry methods.

The detectable label can be directly or indirectly detectable moiety upon hybridization. In other words, a detectable marker has a measurable physical property (e.g., fluorescence or absorption), or to participate in the enzymatic reaction. Direct labeling method, then, the target nucleotide sequence or probe is labeled, to form a hybrid by detecting hybrids markers. Indirect labeling method, then, the second probe is labeled, a hybrid formed by a second hybridization between the detection probe and the second initial hybrid formation.

Labeled probe or a nucleic acid is well known in the art. Suitable labels include fluorophores, chromophores, lumiphores, radioisotopes, electron-dense reagents, the FRET (fluorescence resonance energy transfer), enzymes, and ligands having specific binding substance. Particularly useful enzymatic labels having active groups, such as enzymes; substrate for the enzyme; co-enzymes and enzyme inhibitors (Wisdom, Clin. Chem, 22: 1243 (1976)); fluorophore (Soini and Hemmila, Clin. Chem, 25: 353 (1979)); chromophores comprises a phycobiliprotein, luminescent groups such as chemiluminescent groups and bioluminescence group (Gorus and Schram, Clin. Chem, 25: 512 (1979)); specifically binding ligand, such as protein binding ligand; antigens, and include radioisotopes (e.g., 3H, 35S, 32P, 125I, and 14C) residues. Such a label is detected (e.g., fluorescers, chromophores and radioisotopes) or their reactive or binding properties (e.g. antibodies, enzymes, substrates, co-enzymes and inhibitor) on the basis of their own physical properties based on. Labeled ligand are also useful for the solid phase for the capture oligonucleotide probe (e.g., capture probe). Exemplary labels include biotin (detectable by, and labeled avidin or streptavidin-biotin binding protein) and an enzyme, such as horseradish peroxidase or alkaline phosphatase (enzyme substrate is added by the produces a colored reaction product detectable).

For example, a radioisotope labeled probe or target nucleic acid may be detected by autoradiography. Or with a fluorophore labeled probe or target nucleic acid may be detected by fluorescence meter, as is known in the art. A hapten or ligand (e.g., biotin) labeled nucleic acid, the antibody may be a dye or by adding an antibody to the hapten, or by the addition of the labeled ligand (e.g., biotin) binding protein is detected.

Further, the probe or nucleic acid may be labeled with a structure which is required with other reagents to detect the hybridization. If the label is an enzyme, then the labeled nucleic acid, for example the DNA, eventually placed in a suitable medium up to determine the extent of catalysis. For example, a cofactor-labeled nucleic acid can be detected by this enzyme and substrate for the enzyme is added together, for the enzyme label is a cofactor. Thus, if the enzyme is a phosphatase, the medium may contain nitrophenyl phosphate, and may monitor the number of colors generated by observing nitrophenol. If the enzyme is β-galactosidase, then the medium may contain o-nitro-phenyl-D-galacto-pyranoside, which may be released nitrophenol. Illustrative examples of the latter include, but are not limited to, β-galactosidase, alkaline phosphatase, papain, and peroxidase. For in situ hybridization studies, the final product of the substrate is preferably water-insoluble. Other markers, such as dyes will be apparent to those of ordinary skill in the art is.

Label may be coupled directly to the ligand binding DNA, such as acridine dyes, phenanthridine, phenazine, furocoumarins, phenothiazines and quinolines, such as by direct chemical bond involves direct covalent chemical bonds, or by indirect key such as by the marker incorporated into microcapsules or liposomes, which in turn is connected to the binding ligand. DNA markers are connected to the binding ligand such as a method of embedding compounds is well known in the art and any convenient method can be used. Representative intercalators include mono- or bis-azido aminoalkyl A compound ingot or ethidium, ethidium monoazide ethidium diazide, ethidium dimer azide (Mitchell et al., J. Am. Chem. Soc, 104: 4265 (1982)), 4-azido-7-chloro quinolinyl, 2-azido-fluorenyl, 4′-aminomethyl-4,5′-dimethyl-angelica root element, 4′-carbamoyl group-trioxsalen (4′aminomethyl-4,5′, 8-trimethyl-psoralen), 3-carboxy-5- or 8-amino- or -hydroxy-psoralen. Specific nucleic acid binding compound has the azide (Forster et al. In Nucleic Acid Res., 13: 745 (1985)). Other useful intercalators are formed photoactive furocoumarins (2+2) the pyrimidine ring adduct residues. DNA alkylating agents may also be used as a binding ligand, including, for example bis-chloroethyl amine and epoxides or aziridines, eg, aflatoxins, polycyclic hydrocarbon epoxides, mitomycin, and norphillinA. Particularly useful forms of photoactive agents are embedded azide intercalator. Their reactive nitrenes easily generated at long wavelength ultraviolet or visible light, aryl azide preferentially nitrene insertion reactions, rather than forming their rearrangement products (White et al., Meth. Enzymol, 46: 644 (1977)).

The probe may also be modified for use in a specific format, such as adding 10-100T residues for reverse dot blot, or an albumin binding with bovine serum or immobilized to the magnetic beads.

When hybridization is detected by an indirect detection method, after an initial hybridization between probe and target, and the target or the probe during the hybridization may be added detectably labeled second probe. Optionally, hybridization conditions can be modified after being added to a second probe. After hybridization, unhybridized second probes from an initial probe can be isolated, for example, if the initial probe is immobilized on a solid support can be separated by washing. In the case where the solid support, detection of the bound label on the support indicates the position of the target nucleotide sequence in the sample hybridized to the probe.

Detectably labeled second probe may be specific probe. Optionally, the probes detectably labeled degenerate probes may be, for example, a mixture of total genomic DNA sequences. In the latter case, if the second probe contains the DNA duplex, marking can be achieved by intercalating agents. Preferred binding ligand is DNA intercalation compounds, compounds such as those described above.

The second probe may be a library of random nucleotide probe sequence. The second length of the probe should nucleotide sequence length and composition according to the first probe or target on a solid support to determine which will be detected by the second probe. Such libraries preferably are labeled by the probe light activator 3′ or 5′ end, and a load detecting reagent such as a fluorophore, an enzyme, a dye, luminescent group or other known detectable other end portion is provided.

Sequence used in the preparation of the particular nucleic acid tag can be changed. Thus, for example, an amino-substituted psoralen (psoralen) with the nucleic acid may be first photochemical coupling occurs, the product having an amino side groups, which can be coupled to the label, i.e., DNA binding by labeled ligand to the experimental sample nucleic acid photochemical reaction carried out. Additionally, psoralen can first be coupled to a label such as an enzyme, is then coupled to a nucleic acid.

Advantageously, the DNA binding and ligand binding label is first chemically and then combined with a nucleic acid probe. For example, since biotin carries a carboxyl group, it can be through an amide or ester bond formed with the furanocoumarins, without affecting the photochemical reaction furanocoumarins, biotin or biological activity. Aminomethyl angelica root element, psoralen and phenanthridium derivatives can be similarly connected with the label, phenanthridium halides and derivatives thereof such as aminopropyl chloride A starter (Hertzberg et al, J. Amer., 104: 313 (1982)) such as. Additionally, bifunctional agents such as dithiobis succinimidyl propionate or 1,4-butanediol diglycidyl ether can be used directly to ligand binding to DNA coupled to a marker, wherein the reactants alkylamino having residue, the solvent, the reaction conditions and proportions are carried out in a known manner. Certain bifunctional reagents, possibly glutaraldehyde, may be unsuitable, since when these coupling agents, which may be modified nucleic acid and thus affect the experiment. Routine precautions can be taken to prevent such problems.

Also advantageously, the DNA binding ligands may be attached through a spacer to the marker, which spacer comprises a chain of about 40 atoms, up to, preferably about 2 to 20 atoms, including but not limited to carbon, oxygen, nitrogen, and sulfur. Such spacer groups may include but is not limited to the following members of the multifunctional compound groups: peptide, hydrocarbon, polyalcohol, polyether, polyamine, polyimine and carbohydrate, e.g. -glycyl-glycyl-glycyl- or other oligopeptide, carbonyl dipeptide, and ω-amino-alkyl-carbonyl group or the like. Sugar, polyethylene oxide radicals, glyceryl, pentaerythritol, and the like may be used as the spacer group. The spacer may be directly connected or nucleic acid binding ligand and/markers, or the keys may include a divalent linking group, such as dithiobis succinimidyl propionate, 1,4-butylene alcohol diglycidyl ether, a diisocyanate, carbodiimide, glyoxal, glutaraldehyde, or the like.

A second probe for indirect detection of hybridization may be detected by energy transfer, as in Tyagi and Kramer Nature Biotech's, 14: 303-309, etc. (1996) described “beacon probe” method. Any FRET detection systems known in the art may be used in the method of the present invention. For example, the system may be used Alpha Screen. Alpha Screen technology is an “amplification with the adjacent light-emitting shaped test (Amplified Luminescent Proximity Homogeneous Assay)” method. Once a laser emitting at 680 nm, the oxygen surrounding the photosensitizer will donor beads converted to singlet oxygen. The excited singlet oxygen molecules before diffusion is fast fading about 250 nm (a bead diameter). If donor and acceptor beads in close near the beads, then since biological interactions, singlet oxygen molecules react with the chemiluminescent group in the acceptor beads, which immediately acceptor beads transfer energy to fluorescent beads of the same receptor. The emission wavelength of the fluorescent acceptor is converted into 520-620 nm. Throughout the reaction with a half-life of 0.3 seconds, and therefore may occur in a time-resolved measurement mode. Other exemplary FRET donor/acceptor pair include fluorescein (donor) and tetramethyl rhodamine (acceptor), the effective distance of 55; IAEDANS distance (donor) and fluorescein (acceptor), the effective distance of 46; and fluorescein (donor) and QSY-7 dye (acceptor), the effective distance of 61 (molecular probes).

Quantitative determination of nucleic acid detection can also be carried out in accordance with the present invention. The number of the microarray spots combined with the second probe can be measured and can be related to the number of target nucleic acid in the sample. The diluted sample can be used with the target nucleotide containing known quantities. These steps precise conditions of ordinary skill in the art will be apparent. In microarray analysis, the detectable label is a visually observable (visualized), and evaluated, or the x-ray film or a position adjacent the imager phosphate by the probe array was placed, to identify the binding site of the probe. Fluorescence can be detected by a charge coupled device (CCD) or a laser scanning manner.

Any suitable test specimen samples, including human, animal, or the environment (e.g. soil or water) derived sample, the method of the present invention may be used for analysis. Test sample may comprise body fluids such as urine, blood, semen, cerebral spinal fluid, pus, amniotic fluid, tears or semi-solid or liquid exudates, such as sputum, saliva, inspiratory lung, vaginal or urethral secretions, feces or solid tissue samples such as a biopsy specimen or chorion. Test samples also include samples collected from the skin, genitals or throat swabs.

Test samples can be treated by a variety of methods well known in the art to isolate nucleic acids (see, generally, Ausubel (ed.) Current Protocols in Molecular Biology, Preparation and Analysis of DNA and Preparation and Analysis of RNA, John Wiley & amp; Sons, Inc (2000)).

Purified nucleic acid can be extracted from the sample, and may be determined by photoelectric spectroscopy, or by other assay devices for purification. For nucleic acid amplification of ordinary skill in the art, may be the final amplicon product geted by a variety of amplification methods, such as PCR (polymerase chain reaction), NASBA (based amplified nucleic acid sequence, nucleic Acid Sequence Based amplification), TMA (transcription mediated amplification, transcription mediated amplification) (Kwoh et al., Proc. Natl. Acad. Sci, USA, 86: 1173-1177 (1989)), SDA (strand displacement amplification), tSDA (thermophilic strand displacement amplification, SSSR (self-sustained sequence replication).

In a particular embodiment, the assay sample is of human origin. In still another particular embodiment, the determination of the sputum, urine, blood and tissue sections, food, soil or water samples.

Probe kit of the present invention may be packaged in the kits, preferably with a specification using probes to detect the target gene. Composition of the kit are packaged in a common container, typically including writing method disclosed herein selectively description of specific embodiments. Composition detection method, as described herein, may optionally be included in the kit, e.g., the second probe and/or reagents and methods for accomplishing mark detection (e.g., a radioactive label, an enzyme substrate, antibodies and the like, and the like).

We have two overall design is a design of the different parts of the SARS-CoV-2 genome is a multiplex PCR, and used as a probe to detect the PCR product. The second design is a multiplex PCR for different portions of SARS-CoV-2 genome, and a 70 mer oligonucleotide as a probe for detection.

Based on analysis of selected target genes for SARS-CoV-2 genome, we chose three genes as a target gene. These three genes are polymerase proteins 1A and 1B, spike protein and nucleocapsid proteins. We chose human housekeeping genes of GAPD (glyceraldehyde 3-phosphate dehydrogenase) as a positive control for RNA extraction. We chose Arabidopsis (GenBank Acc: AJ441252) incorporated as a positive control, the human gene or nucleotide sequence is not homologous common pathogens.

Primer and probe design analyzes the three protein of the SARS-CoV-2, and compares their consensus sequence. The requirements of multiplex PCR based on conserved sequences among different genomes, a plurality of designed PCR primers, which have similar Tm values, 1.5 Kb apart at a distance, and between the 200 bp amplification product to 900 bp. Further, it based on an amplification product of each primer pair, designed not to overlap the plurality of oligonucleotides (70 mer). The BLASTN using these primers and a nucleic acid probe with the latest NCBI non-redundant nucleotide database are compared to ensure that the specific primers and probes.

Control is immobilized oligonucleotide probe, which is HEX fluorescent dye labeled on the terminal and, when containing or suspected of containing a sample of SARS-CoV-2 and the kit contacts, the probe does not participate in any hybridization reaction.

Positive control (Arabidopsis thaliana) is an oligonucleotide probe, which is designed in accordance with Arabidopsis (a model biological) gene, when containing or suspected of containing a sample of SARS-CoV-2 and the kit contacts, the probe the needle does not participate in any hybridization reaction. In the process of hybridization reaction may be carried out well and the positive control probe hybridizes to a target is added to the hybridization solution. The positive control signal can be used to monitor the hybridization reaction.

Oligonucleotide probe is the negative control, when containing or suspected of containing a sample of SARS-CoV-2 and the kit contacts, the probe does not participate in any hybridization reaction.

DMSO is blank control point of the solution, which is used to monitor the quality of spotting.

The above example is only for illustrative purposes as an illustration, and not to limit the scope of the invention. Many variations of the examples described above are possible. Since modifications and variations of the above examples are those of ordinary skill in the art it will be apparent, the present invention is limited only by the appended claims be defined later.

### LITERATURE

Lu R, Zhao X, Li J, et al. Genomic characterisation and epidemiology of 2019 novel coronavirus: implications for virus origins and receptor binding. Lancet. 2020; 395:565-574. doi: 10.1016/S0140-6736(20)30251-8.
Zhu N, Zhang D, Wang W, et al. A novel coronavirus from patients with pneumonia in China, 2019 [published online Jan. 24, 2020]. N Engl J Med. 2020. doi:10.1056/NEJMoa2001017.2.
Huang C, Wang Y, Li X, et al. Clinical features of patients infected with 2019 novel coronavirus in Wuhan, China [published online Jan. 24, 2020]. Lancet. 2020. doi:10.1016/S0140-6736(20)30183-5.
Guan W, Ni Y, Hu Y, et al. Clinical characteristics of coronavirus disease 2019 in China. N Engl J Med 2020; 382:1708-1720.1. DOI: 10.1056/NEJMoa2002032.
Li Q, Guan X, Wu P, et al. Early transmission dynamics in Wuhan, China, of novel coronavirus-infected pneumonia [published online Jan. 29, 2020]. N Engl J Med. 2020. doi:10.1056/NEJMoa2001316NEJMoa2001316.
Vital Surveillances: the epidemiological characteristics of an outbreak of 2019 novel coronavirus diseases (COVID-19)-China, 2020. China CDC Weekly. 2020, 2(8):113-122.
Wrapp D, Wang N, Corbett K S, et al. Cryo-EM structure of the 2019-nCoV spike in the prefusion conformation [published online Feb. 19, 2020]. Science. 2020. doi: 10.1126/science.abb2507.
Hoffmann M, Kleine-Weber H, Schroeder S, et al. SARS-CoV2cell entry depends on ACE2 and TMPRSS2 and is blocked by a clinically-proven protease inhibitor. Cell. 2020. doi: 10.1016/j.cell.2020.02.052.
Hamming I, Timens W, Bulthuis M L et al. Tissue distribution of ACE2 protein, the functional receptor for SARS coronavirus. A first step in understanding SARS pathogenesis. J Pathol. 2004; 203:631-7.
World Health Organization. Clinical management of severe acute respiratory infection when novel coronavirus (nCoV) infection is suspected. Interim guidance on Jan. 12, 2020. (accessed Feb. 15, 2020).
Pan X, Chen D, Xia Y, et al. Asymptomatic cases in a family cluster with SARS-CoV2 infection [published online Feb. 19, 2020]. Lancet Infect Dis. 2020. doi: 10.1016/S1473-3099(20)30114-6.
Rothe C, Schunk M, Sothmann P, et al. Transmission of 2019-nCoV infection from an asymptomatic contact in Germany [published online Jan. 30, 2020]. N Engl J Med. 2020. doi: 10.1056/NEJMc2001468.
Laboratory testing for 2019 novel coronavirus (2019-nCoV) in suspected human cases. Interim guidance. Jan. 17, 2020. https://www.who.int/publications-detail/laboratory-testing-for-2019-novel-coronavirus-in-suspected-human-cases-20200117 (accessed Feb. 21, 2020).
Xu Y, Li Y R, Zeng Q, et al. Clinical characteristics of SARS-CoV-2 pneumonia compared to controls in Chinese Han population. www.medrxiv.org [posted Mar. 10, 2020]. https://www.medrxiv.org/content/10.1101/2020.03.08.20031658v1.
Cunningham-Rundles C, Ponda P P. Molecular defects in T- and B-cell primary immunodeficiency diseases. Nat Rev Immunol. 2005; 5:880-92.
Pirsch J D, Maki D G. Infectious complications in adults with bone marrow transplantation and T-cell depletion of donor marrow. Increased susceptibility to fungal infections. Ann Intern Med 1986; 104:619-31.
Gu J, Korteweg. Pathology and pathogenesis of severe acute respiratory syndrome. Am J Pathol. 2007; 170: 1136-47.
Xu Z, Shi L, Wang Y, et al. Pathological findings of COVID-19 associated with acute respiratory distress syndrome [published online Feb. 18, 2020]. Lancet Respir Med. 2020. doi: 10.1016/S2213-2600(20)30076-X.
Zhou P, Yang X L, Wang X G et al. A pneumonia outbreak associated with a new coronavirus of probable bat origin [published online Feb. 3, 2020]. Nature. 2020. doi: 10.1038/s41586-020-2012-7.
Liu B, Zhang X, Deng W, et al. Severe influenza (H1N1)pdm09 infection induces thymic atrophy through activating innate CD8⁺CD44^{hi} T cells by upregulating IFN-γ. Cell Death and Disease. 2014. 5:e1440.
Cheng Y D, Zhao H, Song P X, et al. Dynamic change of lymphocyte counts in adult patients with severe pandemic H1N1 influenza A. J Infect Public Health. 2019; 12(6):878-83.
Virk R K, Jayakumar J, Mendenhall I H, et al. Divergent evolutionary trajectories of influenza B viruses underlie their contemporaneous epidemic activity. PNAS. 2020; 117(1): 619-628.

1. A kit for detecting a cause of severe acute respiratory syndrome coronavirus 2 (SARS-CoV-2) and non-biological SARS-CoV-2 co-infection, wherein the gene mutation sequence is selected from the group consisting of COIN-CoV2-241, COIN-CoV2-3037, COIN-CoV2-14408, COIN-CoV2-28144, COIN-CoV2-23403, COIN-CoV2-28881, COIN-CoV2-28882, COIN-CoV2-28883, COIN-CoV2-17747, COIN-IFA-238 and COIN-IFB-002 in Table 1, said kit comprising a support, suitable for use in nucleic acid hybridization, the support a may be immobilized oligonucleotide probes with SARS-CoV-2 genome nucleotide sequence complementary to the nucleotide sequence comprising at least 10 nucleotides, and one or more of the following immobilized oligonucleotide acid probe: a) a nucleotide sequence of an oligonucleotide probe organism infection with COVID-19-like symptoms lead to non-SARS-CoV-2 complementary to, the nucleotide sequence comprises at least 10 nucleotides; b) with a non-SARS-CoV-2 coronaviridae viral nucleotide sequence complementary to an oligonucleotide probe, the nucleotide sequence comprises at least 10 nucleotides; or c) with an assay for identifying single nucleotidepoly morphism (SNPs).

2. The kit according to claim 1, said kit comprising a support, suitable for use in nucleic acid hybridization, the support may be immobilized at least two different nucleotides at least two SARS-CoV-2 genome acid sequence complementary to the oligonucleotide probes, each of the two different nucleotide sequences comprising at least 10 nucleotides.

3. The kit of claim 2, wherein at least two different nucleotide sequences of SARS-CoV-2 genome comprises: a nucleotide sequence of a) is located in a conserved region of SARS-CoV-2 genome of at least 10 nucleotides, located in the variable region and a SARS-CoV-2 genome a nucleotide sequence of at least 10 nucleotides; or b) is located in a SARS-CoV-2 genome encoding the structural protein genes of at least 10 nucleotides nucleosides acid sequence, a gene coding for a protein and a nonstructural SARS-CoV-2 genome of at least 10 nucleotides of the nucleotide sequence.

4. The kit of claim 2, said kit further comprising: a) the following three oligonucleotide probes, at least one of: an immobilized control probes, the probe is labeled, or when comprising SARS-CoV-2 or suspected of containing SARS-CoV-2 infection in a biological sample in contact with the kit, the probe does not participate in any hybridization reaction; a positive control probe, which sequence is any SARS-CoV-2 or SARS-CoV-2 infection organisms are not complementary, but with a sequence contained in the sample or in the SARS-CoV-2 infection in the organism is not found complementary; a negative control probe which does not comprise any nucleotide sequence in the sample complementary; and b) a blank point.

5. The kit of claim 2 in which the sequence in claim 2, said kit comprising at least two oligonucleotide probes, respectively, and contain two different nucleotide sequence complementary to at least 10 nucleotides, those are located in a conserved region of the genome of SARS-CoV-2, a protein-encoding gene located SARS-CoV-2 genome or in the SARS-CoV-2 genome encoding a nonstructural protein gene.

6. The kit as claimed in claim 2, wherein SARS-CoV-2 structural protein coding gene of the genome encoding the spike protein (S), a small envelope protein gene (E) or nucleocapsid protein (N).

7. The kit of claim 2, wherein the variable region of SARS-CoV-2 genome is located in a region of SARS-CoV-2 spike protein (S) gene.

8. The kit of claim 2, the kit comprises four oligonucleotide probes of at least two of the following: two located SARS-CoV-2 replicase least 10 nuclear 1A or 1B gene two different nucleotide sequences complementary to the nucleotide of the oligonucleotide probe, an oligonucleotide probe and a nucleotide sequence of at least 10 nucleotides is located in the SARS-CoV-2 N gene complementary to, and oligonucleotide probes complementary to a nucleotide sequence of at least 10 nucleotides of the SARS-CoV-2 S gene located in.

9. The kit of claim 8, wherein in one of two different nucleotide sequences of SARS-CoV-2 replicase 1A or 1B gene comprises a nucleotide sequence having the following features replicase and the table 1 in a nucleotide sequence or a complementary strand of 1A or 1B; or b) comprises a replicase and the nucleotide sequence of the nucleotide sequence in table 1 or its complementary strand of 1A or 1B having at least 90% identity.

10. The kit of claim 9, wherein the SARS-CoV-2 replication located in one or two different enzyme 1A 1B gene nucleotide sequences comprising a nucleotide sequence shown in Table 1.

11. The kit of claim 8, wherein the nucleotide sequence is located in the SARS-CoV-2 N gene comprises a nucleotide sequence having the following features: a) N gene at high stringency and the nucleotide sequence or its complementary strand; or b) or its complementary strand and the nucleotide sequence comprising a nucleotide sequence in the N gene having at least 90% identity.

12. The kit of claim 11, wherein the nucleotide sequence is located in the SARS-CoV-2 N gene comprises a nucleotide sequence.

13. The kit of claim 8, wherein the nucleotide sequence is located in the SARS-CoV-2 S gene comprises a nucleotide sequence having the following features the nucleotide sequence of the gene or a complementary strand; or b) and comprises a nucleotide sequence or a complementary strand shown in table 1 S gene nucleotide sequence having at least 90% identity.

14. The kit of claim 13, wherein the nucleotide sequence is located in the SARS-CoV-2 S gene comprises a nucleotide sequence.

15. The kit 4 wherein the immobilized control probe label is selected from a chemical as claimed in claim, enzymatic, immunochemical, radiolabeled, fluorescent, luminescent and a FRET label.

16. The kit of claim 4, wherein the non-incorporated into the sequence of SARS-CoV-2 in the sample is detected.

17. The kit of claim 16, wherein the non-SARS-CoV-2 sequences derived from Arabidopsis incorporated sequence.

18. The kit of claim 8, the kit comprises two oligonucleotide probes with two different nucleotide sequences of SARS-CoV-2 replicase 1A or 1B complementary gene; and a SARS-CoV-2 N gene in the nucleotide sequence complementary to an oligonucleotide probe; oligonucleotide probe complementary to a section of the S gene of SARS-CoV-2 in a nucleotide sequence; an immobilized control probe, the probe is labeled, when containing or suspected of containing a SARS-CoV-2 or SARS-CoV-2 infection in a biological sample in contact with the kit, the probe does not participate in any hybridization reaction; a positive control probe, which SARS-CoV-2 or any sequence of SARS-CoV-2 infection organism not complementary, but one contained in the sample, or SARS-CoV-2 sequences SARS-CoV-2 infection in the organism is not found complementary; a negative control probe, which not complementary to any nucleotide sequence contained in the sample.

19. The kit of claim 18, said kit comprising a plurality of probe points as follows: two and two different nucleotide sequences of SARS-CoV-2 replicase 1B gene oligonucleotide probes complementary to needle, a nucleotide sequence complementary to the N gene of SARS-CoV-2 in the oligonucleotide probe and a complementary nucleotide sequence of the S gene of SARS-CoV-2 in the oligonucleotide probes, a immobilized control probes, a positive control and a negative control probe.

20. The kit of claim 4, wherein the at least one oligonucleotide probe 5′ end a poly-dT area, to enhance its immobilized on a support.

21. The kit of claim 2, wherein the at least one oligonucleotide probe and the nucleotide sequence of a SARS-CoV-2 genome highly expressed complementary.

22. The kit of claim 1, wherein the COVID-19-like symptoms, leading to a non-biological SARS-CoV-2 infection is selected from influenza A virus or influenza B virus.

23. The kit of claim 1, wherein said support comprises a material selected from silicon, plastic, glass, ceramics, rubber and the surface of the polymer surface.

24. A method for detecting SARS-CoV-2 in the sample and non-biological SARS-CoV-2 infection, the method comprising: a) providing a kit as claimed in claim 1; b) containing or suspected of containing non-SARS-CoV-2 and SARS-CoV-2 infection in a biological sample a nucleotide sequence of the kit contacts under conditions suitable for nucleic acid hybridization; and c) evaluating the nucleotide sequence of the SARS-CoV-2 or the non-SARS-CoV-2 infection organism, if the if present in the sample, and the nucleotide sequence of the genome of SARS-CoV-2 is complementary to the oligonucleotide probe or a nucleotide sequence complementary to the non-SARS-CoV-2 infection in the genome of oligonucleotide hybrid complexes formed between the nucleotide probe is detected so long as one or both of the hybridization complex indicates the presence on the SARS-CoV-2 and/or the non-SARS-CoV-2 infection in the biological sample.

25. The method of claim 24, wherein the detection of SARS-CoV-2 by the following steps: a) providing a kit as claimed in claim 2; b) with a sample containing or suspected of containing the nucleotide sequence of SARS-CoV-2 in a suitable nucleic acid hybridization contacting the kit under conditions; and c) evaluating the SARS-CoV-2 nucleotide sequences, if present in the sample and then two different nucleotide sequence of the genome of SARS-CoV-2 were complementary to the hybridization complexes formed between the at least two oligonucleotide probes to determine the presence in the sample of SARS-CoV-2, or a number or not, so long as the detection of one or both of the hybridization complexes to show that the presence of SARS-CoV-2 in the sample.

26. The method of claim 24, wherein the detection of SARS-CoV-2 by the following steps: a) providing a kit as claimed in claim 3; b) with a sample containing or suspected of containing the nucleotide sequence of SARS-CoV-2 in a suitable nucleic acid hybridization contacting the kit under conditions; and c) evaluating the SARS-CoV-2 nucleotide sequences, if present in said sample; hybridization complexes are formed, and the following i) and ii) between, i) are respectively located SARS-CoV-2 nucleotide sequence conserved region of the genome of the oligonucleotide probes complementary to, the nucleotide sequences of the variable and a region of the SARS-CoV-2 genome of oligonucleotide probes complementary to the or ii) a nucleotide sequence is located SARS-CoV-2 genome encoding the structural protein gene complementary to said oligonucleotide probe, and a non-structural protein-encoding gene nucleotide located SARS-CoV-2 genome of sequence complementary to an oligonucleotide probe to determine the presence or absence of SARS-CoV-2 in the sample number, so long as the detection of one or both of the hybridization complex indicates the presence in the sample just as said SARS-CoV-2.

27. The method of claim 24, wherein the detection of SARS-CoV-2 by the following steps: a) providing a kit as claimed in claim 4; b) with a sample containing or suspected of containing the nucleotide sequence of SARS-CoV-2 in a suitable nucleic acid hybridization contacting the kit under conditions; and c) evaluation: i), if present in the sample in the nucleotide sequence of the SARS-CoV-2; and an oligonucleotide complementary to a conserved region of the genome of SARS-CoV-2 nucleotide sequences nucleotide probe and the hybridization complex formed between the nucleotide sequence of the variable region of SARS-CoV-2 genome complementary oligonucleotide probes; ii) comprising markers of the immobilized control probe or relates to the positive control probe and/or negative control probe hybridization complex; and iii) a point on the blank signal, to determine the presence in the sample of SARS-CoV-2, or a number or not.

28. The method of claim 27, wherein the kit comprises two located SARS-CoV-2 replicase 1A or 1B two different gene nucleotide sequences complementary to the oligonucleotide probes, located at a the nucleotide sequence of the SARS-CoV-2 N gene complementary oligonucleotide probes, one complementary to the nucleotide sequence of SARS-CoV-2 S located gene oligonucleotide probes, a probe-immobilized control pin, a positive control and a negative control probe, when determined that there may be a case where the occurrence of SARS-CoV-2: a) located using SARS-CoV-2 replicase 1A or 1B and two different gene nucleotide sequence of two oligonucleotide probes complementary to, the nucleotide sequence is located in the SARS-CoV-2 N gene oligonucleotide probes complementary to and/or located at nucleotides of SARS-CoV-2 S gene sequence complementary to an oligonucleotide probe at least one oligonucleotide probe, a positive hybridization signal is detected; b) with immobilized control probes, positive signal is detected; c) with the positive control probe, detection to positive hybridization signals; d) using a negative control probe positive hybridization signals cannot be detected; and e) at point blank, not detect positive hybridization signals.

29. The method of claim 28, wherein the nucleotide sequence is located in two different SARS-CoV-2 replicase 1A or 1B, two oligonucleotide probes complementary to at least one oligonucleotide probe, or a nucleotide sequence is located N gene of SARS-CoV-2 complementary oligonucleotide probes, positive hybridization signals can be detected; the use of the nucleotide sequence is located in the SARS-CoV-2 S gene complementary oligonucleotide probe positive hybridization signals cannot be detected, suggesting that the mutation of SARS-CoV-2.

30. The method as claimed in claim 24, wherein the kit as claimed in claim 21, which is used for the early diagnosis of patients with SARS-CoV-2.

31. The method as claimed in claim 30, wherein the early SARS-CoV-2 infected patients have been infected with about less than one day to about three days of SARS-CoV-2.

32. A method as claimed in claim 23, the method is used to determine whether a subject is infected with SARS-CoV-2 and/or cause a COVID-19-like symptoms of SARS-CoV-2 infection of non-biological.

33. The method of claim 23, wherein the nucleotide sequence of the SARS-CoV-2 or SARS-CoV-2 infection organism is SARS-CoV-2 or SARS-CoV-2 genome sequence of the infecting organism, or a SARS-CoV-2 genome sequence of an RNA extracted from the amplified DNA sequences, or SARS-CoV-2 infection in the extracted genomic sequence of an organism.

34. The method of claim 33, wherein the SARS-CoV-2 RNA genome sequence using the QIAamp Viral RNA kit, Chomczynski-Sacchi technique or TRIzol extraction from the SARS-CoV-2 infected cells.

35. The method of claim 33, wherein the SARS-CoV-2 RNA genome sequence using a QIAamp Viral RNA extraction kit from SARS-CoV-2 infected cells.

36. The method of claim 24, wherein the SARS-CoV-2 or SARS-CoV-2 infection in the organism genome sequence lymphocytes extracted from sputum or saliva sample, a blood sample.

37. The method of claim 24, wherein the SARS-CoV-2 or IFA, IFB infection or genomic sequence from the human organism, mice, dogs, rats, cats, horses, birds, soil, water, air, nasopharynx, oral pharynx, trachea, bronchaleolar lavage, pleural fluid, urine, feces, conjunctiva, tissue extracts.

38. The method of claim 33, wherein the SARS-CoV-2 or IFA or IFB genome sequence infecting organism by PCR amplification.

39. A method as claimed in claim 38, wherein the label is incorporated into the amplified DNA sequences during PCR.

40. The method as claimed in claim 38, wherein PCR involves conventional PCR, multiplex PCR, nested PCR or RT-PCR.

41. The method of claim 38, wherein PCR involves a two-step nested PCR, the first step is a RT-PCR, and the second step is a conventional PCR.

42. The method of claim 38, wherein the PCR comprises a step multiplex RT-PCR, which uses multiple 5′ and 3′ specific primers, wherein each primer comprises a specific thereto amplified target sequence the specific sequence and a common sequence, and a 5′ and 3′ universal primers, wherein the 5′ universal primer with a 5′ specific primer consensus sequence is complementary to and a common sequence ‘universal primer 3’ specific primer complementary to, wherein in PCR, 5′ and 3′ universal primer concentrations are equal to or higher than the concentration of 5′ and 3′ specific primers.

43. The method of claim 42, wherein the 3′ universal primer and/or a 5′ universal primer is labeled.

44. The method as claimed in claim 43, wherein the label is a fluorescent label.

45. The method as claimed in claim 38, wherein the multiplex PCR comprises a nested PCR.

46. The method of claim 38, wherein the PCR below 19-21 primer pair at least one primer pair.
